(19) United States
(12) Patent Application Publication
(10) Pub. No.: US 2016/0101086 A1
(43) Pub. Date: Apr. 14, 2016

(54) METHODS OF TREATING MUSCULAR DYSTROPHY

(71) Applicant: Salk Institute for Biological Studies, La Jolla, CA (US)

(72) Inventors: Michael Downes, San Diego, CA (US); Ruth T. Yu, La Jolla, CA (US); Vihang A. Narkar, Houston, TX (US); Ronald M. Evans, La Jolla, CA (US)

(73) Assignee: Salk Institute for Biological Studies, La Jolla, CA (US)

(21) Appl. No.: 14/972,711

(22) Filed: Dec. 17, 2015

Related U.S. Application Data
(63) Continuation of application No. PCT/US2014/042885, filed on Jun. 18, 2014.
(60) Provisional application No. 61/836,592, filed on Jun. 18, 2013.

(51) Int. Cl. A61K 31/42; G01N 33/74

(52) U.S. Cl. CPC A61K 31/42; G01N 33/743; G01N 2333/723; G01N 2800/52

(57) ABSTRACT

Disclosed herein are methods of improving muscular health, such as enhancing muscle regeneration, maintenance, or repair. In some examples, the method includes administering to the subject an effective amount of an agent capable of increasing activity or expression of estrogen receptor-related gamma (ERRγ), related receptors ERRα or β, or ERR-regulated metabolic and angiogenic genes, thereby enhancing muscle regeneration, maintenance, or repair. In some examples, the methods are utilized to treat a subject with one or more signs or symptoms of muscular dystrophy, such as, but not limited to Duchenne muscular dystrophy. In some examples, the disclosed methods further include selecting a subject in need of enhancing muscle regeneration, maintenance, or repair.

### CROSS REFERENCE TO RELATED APPLICATIONS

This is a continuation application of PCT Patent Application No. PCT/US2014/042885, filed Jun. 18, 2014, which claims the benefit of U.S. Provisional Patent Application No. 61/836,592, filed Jun. 18, 2013, both of which applications are hereby incorporated by reference in their entirety.

### ACKNOWLEDGMENT OF GOVERNMENT SUPPORT

This invention was made with government support under DK-057978 awarded by the National Institutes of Health. The government has certain rights in the invention.

### FIELD

This application relates to muscular dystrophy and in particular, to methods of treating muscular dystrophy, such as Duchenne muscular dystrophy (DMD), by modulating, such as increasing, expression of estrogen receptor-related gamma (ERRγ).

### BACKGROUND

Duchenne muscular dystrophy (DMD) is an inherited X-linked muscle degenerative disease that affects approximately 1 in 3500 boys and is caused by inactivating mutations in structural protein dystrophin. Loss of dystrophin, and in turn dystrophin-associated glycoprotein (DAG) complex, primarily results in sarcolemmal fragility leading to muscle wasting as well as increased susceptibility to contraction or activity-induced damage and fatigue. High rate of early mortality in DMD is due to respiratory or cardiac muscle fatigue and failure. One of the most extensively explored approaches to treat DMD has been to replace functional dystrophin or to up-regulate a dystrophin-related surrogate protein utrophin using genetic (e.g., muscle transgenesis), molecular (e.g., minidystrophins or utrophin gene transfer), pharmacological (e.g., synthetic transcriptional regulators), and integrative (e.g., transplantation of healthy tissue in dystrophic muscle) strategies. This approach has been rooted in the rationale that restoring DAG can reverse sarcolemmal fragility and abate myodystrophy. However, the strategy to correct the structural fragility by dystrophin replacement has met with challenges and limited clinical success. Consequently, DMD has remained incurable, warranting preclinical studies aimed at discovering alternative therapeutic options.

### SUMMARY

Disclosed herein are methods of improving muscular health, such as enhancing muscle regeneration, maintenance, or repair. In some examples, the method includes administering to the subject an effective amount of an agent capable of increasing activity or expression of ERRγ, related receptors ERRα or β, or ERR-regulated metabolic and angiogenic genes, thereby enhancing muscle regeneration, maintenance, or repair. In some examples, the methods are utilized to treat a subject with one or more signs or symptoms of muscular dystrophy, such as, but not limited to DMD. In some examples, the disclosed methods further include selecting a subject in need of enhancing muscle regeneration, maintenance, or repair.

In some examples, the present disclosure also provides a method for increasing muscle regeneration in a subject. For example, geriatric subjects, subjects suffering from muscle disorders, and subjects suffering from muscle injury, including activity induced muscle injury, such as injury caused by exercise, may benefit from this embodiment.

In some examples of the disclosed method, an agent capable of increasing ERRγ is administered in a preventative manner, such as to prevent or reduce muscular damage or injury (such as activity or exercise induced injury). For example, geriatric subjects, subjects prone to muscle damage, or subjects at risk for muscular injury, such as athletes, may be treated in order to eliminate or ameliorate muscular damage, injury, or disease.

In further embodiments, the method of the present disclosure includes administering one or more additional pharmacological substances, such as a therapeutic agent. In some aspects, the additional therapeutic agent enhances the therapeutic effect of the agent capable of increasing ERRγ, related receptors ERRα or β, or ERR-regulated metabolic and angiogenic genes activity or expression.

In some embodiments, the disclosed methods are utilized to treat a muscular disease. In some embodiments, the muscular disease is a muscular dystrophy. In some specific embodiments, the muscular dystrophy is Duchenne muscular dystrophy, Becker muscular dystrophy, limb-girdle muscular dystrophy, congenital muscular dystrophy, facioscapulohumeral muscular dystrophy, myotonic muscular dystrophy, oculopharyngeal muscular dystrophy, distal muscular dystrophy, and/or Emery-Dreifuss muscular dystrophy. In some embodiments, the muscular dystrophy is Duchenne muscular dystrophy. In some embodiments, the disease is a demyelinating disease. In some further embodiments, the demyelinating disease is multiple sclerosis, Charcot-Marie-Tooth disease, Pelizaeus-Merzbacher disease, encephalomyelitis, neuromyelitis optica, adrenoleukodystrophy, and/or Guillian-Barre syndrome.

The foregoing and other features and advantages of the disclosure will become more apparent from the following detailed description, which proceeds with reference to the accompanying figures.

### BRIEF DESCRIPTION OF THE DRAWINGS

FIGS. 1A-1E illustrate metabolic and angiogenic biomarker expression in muscle. Following parameters were measured in the wild-type (C57), mdx, and mdx-ERRγ transgenic (mdx-tg) mice. A, B). Relative expression of metabolic (A) and angiogenic (B) genes in quadriceps (Quad), TA, and soleus (Sol). C, D). Protein expression levels of metabolic biomarkers (C) and VEGFa (D) in gastrocnemius (Gastroc; n=3, each sample pooled from 2 mice). E) Average spontaneous ambulatory activity during daytime and nighttime (n=4). In A, B, and D, data are presented as means±sd from n=6 mice/group. *P<0.05 between mdx-tg and mdx mice, #P<0.05 between mdx or mdx-tg and C57 mice; 1-way ANOVA.

FIGS. 2A-2D illustrate oxidative capacity in the dystrophic muscle. Oxidative capacity biomarkers were measured in the wild-type (C57), mdx, and mdx-tg mice. A) Representative images of SDH staining in superficial and deep TA depicting oxidative capacity (dark stained myofibers; n=6). Scale bar=100 p.m. B) Representative images of cross-sectional staining for type IIA and IIB myofibers (n=6). Scale bar=100 p.m. C) Quantification of mitochondrial biogenesis in gastrocnemius and quadriceps (n=6). *P<0.005; 1-way ANOVA. D) Exercise tolerance (swimming) test showing time to exhaustion (n=6). *P<0.005; 1-way ANOVA. #Test in wild-type (C57) mice was terminated at 90 minutes without reaching exhaustion. In C and D, data are presented as means±sd.

FIGS. 3A-3C illustrate vasculature in the dystrophic muscle. A) Representative images of vascular mapping in the TA visualized by microfil perfusion in wild-type (C57), mdx, and mdx-tg mice (n=6). B) Angiogenic properties in the dystrophic TA measured using capillary staining (top panel) and microsphere perfusion (bottom panel). Scale bar=100 μm (n=6). C) Laser Doppler blood flow in the TA and gastrocnemius. Data are presented as means±sd from n=10 mice/group. *P<0.001; 1-way ANOVA.

FIGS. 4A-4C illustrate basal muscle damage in mdx mice. A) Serum CK levels in C57, mdx, and mdx-tg mice data are presented as means±sd from n=12 mice/group. *P<0.01; 1-way ANOVA. B) Representative images of Evans blue dye (EBD) infiltration in damaged myofibers of the TA and gastrocnemius from mdx and mdx-tg mice (n=6). Scale bar=100 p.m. C) Representative images of central nuclei (blue) in the TA cryosections from the mdx and mdx-tg mice. Myofiber boundary is stained. Scale bar=100 p.m. D) Quantification of the centralized nuclei. Data are presented as means±sd from n=10 mice/group and 5 images/muscle. *P<0.001; Student's t test.

FIGS. 5A-5C illustrate exercise-induced muscle damage and hypoxia in mdx mice. A) Serum CK levels in mdx and mdx-tg mice preexercise and 1 hour postexercise. Mice were subjected to 4 consecutive 30-min swimming bouts, and serum was collected on day 4. Data are presented as means±sd from n=15 mice/group. *P<0.001; 2-way ANOVA. B) Representative images of postexercise EBD infiltration in damaged myofibers from the TA and gastrocnemius (n=6). C) Postexercise hypoxia in TA and gastrocnemius detected using hypoxyprobe. Scale bar=100 μm (representative from n=6 mice).

FIGS. 6A-6E illustrate the effect of ERRγ on DAG. A-D) Representative images of dystrophin (A), utrophin (B), α-sarcoglycan (C), and α-sarcoglycan (D) staining in cryosections of TA obtained from C57, mdx, and mdx-tg mice (n=6). Scale bar=100 p.m. E) Quantification of utrophin and α/β-sarcoglycan immunofluorescence staining in superficial TA muscles. #P<0.001 vs. C57; 1-way ANOVA.

FIG. 7 illustrates transcriptional changes in mdx and mdx-tg T.A. muscle depots. Myogenesis and Myopathy PCR array data comparing fold change in gene expression in mdx and mdx-tg T.A. vs. wild type T.A is provided in tabular format.

### DETAILED DESCRIPTION

### I. Introduction

Treatment of DMD by replacing mutant dystrophin or restoring dystrophin-associated glycoprotein complex (DAG) has been clinically challenging. The inventors have discovered that the expression of ERRγ, and its metabolic and angiogenic targets are down-regulated (50-85%) in skeletal muscles of mdx mice (DMD model) versus wild-type mice. Correlatively, oxidative myofibers, muscle vasculature, and exercise tolerance (33%) were decreased in mdx versus wild-type mice. Overexpressing ERRγ selectively in the dystrophic muscles of the mdx mice restored metabolic and angiogenic gene expression compared with control mdx mice. Further, ERRγ enhanced muscle oxidative myofibers, vasculature, and blood flow (by 33-66%) and improved exercise tolerance (by 75%) in the dystrophic mice. Restoring muscle ERRγ pathway ameliorated muscle damage and also prevented DMD hallmarks of postexercise muscle damage, hypoxia, and fatigue in mdx mice. Notably, ERRγ did not restore sarcolemmal DAG complex, which is thus dispensable for antidystrophic effects of ERRγ. These studies demonstrate that ERRγ signaling is decreased in the dystrophic skeletal muscle lacking dystrophin and demonstrate that ERRγ activation mitigates DMD.

Based upon the discovery that ERRγ-dependent metabolic and angiogenic gene program is defective in DMD, disclosed herein are methods which restore this pathway that can be used to treat muscular dystrophy.

### II. Terms

In order to facilitate an understanding of the disclosure presented, the following explanations are provided.

Unless otherwise explained, all technical and scientific terms used herein have the same meaning as commonly understood by one of ordinary skill in the art to which this disclosure belongs. In case of conflict, the present specification, including explanations of terms, will control. The singular terms “a,” “an,” and “the” include plural referents unless context clearly indicates otherwise. Similarly, the word “or” is intended to include “and” unless the context clearly indicates otherwise. The term “comprising” means “including;” hence, “comprising A or B” means including A or B, or including A and B. All numerical ranges given herein include all values, including end points (unless specifically excluded) and any and all intermediate ranges between the endpoints.

Administration: The introduction of a composition into a subject by a chosen route. Administration can be systemic or local. For example, if the chosen route is intravenous, the composition is administered by introducing the composition into a vein of the subject, and if the chosen route is intramuscular, the compositing is administered by introducing the composition into a muscle.

Agent: Any protein, nucleic acid molecule, compound, small molecule, organic compound, inorganic compound, or other molecule of interest. Agent can include a therapeutic agent, a diagnostic agent or a pharmaceutical agent. A therapeutic or pharmaceutical agent is one that alone or together with an additional compound induces the desired response (such as inducing a therapeutic or prophylactic effect when administered to a subject). In some examples, an agent is an ERRγ agonist. In some examples, an agent is an ERRγ antagonist.

Antibody: A polypeptide substantially encoded by an immunoglobulin gene or immunoglobulin genes, or fragments thereof, which specifically binds and recognizes an analyte (antigen). In one example, the antibody specifically binds to ERRγ. Immunoglobulin genes include the kappa, lambda, alpha, gamma, delta, epsilon and mu constant region genes, as well as the myriad immunoglobulin variable region genes.

Antibodies exist, for example as intact immunoglobulins and as a number of well characterized fragments produced by digestion with various peptidases. For instance, Fabs, Fvs, and single-chain Fvs (SCFvs) that bind to ERRγ or fragments thereof would be ERRγ specific binding agents. This includes intact immunoglobulins and the variants and portions of them well known in the art, such as Fab′ fragments, F(ab)′₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (such as humanized murine antibodies), heteroconjugate antibodies such as bispecific antibodies). See also, Pierce Catalog and Handbook, 1994-1995 (Pierce Chemical Co., Rockford, Ill.); Kuby, J., Immunology, 3rd Ed., W.H. Freeman & Co., New York, 1997.

Functional fragments of antibodies specifically bind the antigen of interest, and include: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)2, the fragment of the antibody obtained by treating whole antibody with the enzyme pepsin without subsequent reduction; (4) F(ab′)2, a dimer of two Fab′ fragments held together by two disulfide bonds; (5) Fv, a genetically engineered fragment containing the variable region of the light chain and the variable region of the heavy chain expressed as two chains; and (6) single chain antibody (“SCA”), a genetically engineered molecule containing the variable region of the light chain, the variable region of the heavy chain, linked by a suitable polypeptide linker as a genetically fused single chain molecule. The term “antibody,” as used herein, also includes antibody fragments either produced by the modification of whole antibodies or those synthesized de novo using recombinant DNA methodologies.

Typically, a naturally occurring immunoglobulin has heavy (H) chains and light (L) chains interconnected by disulfide bonds. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs.” The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. Light chain CDRs are sometimes referred to as CDR L1, CDR L2, and CDR L3. Heavy chain CDRs are sometimes referred to as CDR H1, CDR H2, and CDR H3.

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. These fused cells and their progeny are termed “hybridomas.” In one example, monoclonal antibodies to EERγ are employed. For example, the monoclonal antibody can be a ERRγ monoclonal antibody (such as Santa Cruz #sc-393969 and R&D Systems Clone H6812 (#PP-H6812-00) monoclonal antibodies).

Monoclonal antibodies include humanized monoclonal antibodies. A “humanized” immunoglobulin is an immunoglobulin including a human framework region and one or more CDRs from a non-human (such as a mouse, rat, or synthetic) immunoglobulin. The non-human immunoglobulin providing the CDRs is termed a “donor,” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, such as at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (for example, see U.S. Pat. No. 5,585,089). Specific examples of humanized antibodies include those that are commercially available from Zenapax (Roche) and Humira (Abbott).

Contacting: Placement in direct physical association; includes both in solid and liquid form. Contacting can occur in vitro with isolated cells or in vivo by administering the agent to a subject.

Control: A reference standard. A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

Degenerate variant and conservative variant: A polynucleotide encoding a polypeptide or an antibody that includes a sequence that is degenerate as a result of the genetic code. For example, a polynucleotide encoding a polypeptide antagonist or a neutralizing antibody that includes a sequence, wherein the polynucleotide is degenerate as a result of the genetic code. There are 20 natural amino acids, most of which are specified by more than one codon. Therefore, all degenerate nucleotide sequences are included as long as the amino acid sequence of the polypeptide encoded by the nucleotide sequence is unchanged. Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified within a protein encoding sequence, the codon can be altered to any of the corresponding codons described without altering the encoded protein. Such nucleic acid variations are “silent variations,” which are one species of conservative variations. Each nucleic acid sequence herein that encodes a polypeptide also describes every possible silent variation. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

Furthermore, one of ordinary skill will recognize that individual substitutions, deletions or additions which alter, add or delete a single amino acid or a small percentage of amino acids (for instance less than 5%, in some embodiments less than 1%) in an encoded sequence are conservative variations where the alterations result in the substitution of an amino acid with a chemically similar amino acid.

Conservative amino acid substitutions providing functionally similar amino acids are well known in the art. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Not all residue positions within a protein will tolerate an otherwise “conservative” substitution. For instance, if an amino acid residue is essential for a function of the protein, even an otherwise conservative substitution may disrupt that activity. Thus, a conservative substitution does not alter the basic function of a protein of interest.

Decrease: To reduce the quality, amount, or strength of something. In one example, a therapy decreases one or more symptoms associated with the muscular dystrophy, for example as compared to the response in the absence of the therapy.

Detect: To determine if a molecule (such as a signal or particular nucleotide nucleic acid probe, amino acid, or protein) is present or absent. In one example, expression of ERRγ is detected. In some examples, this can further include quantification.

Estrogen receptor-related γ (ERRγ): Estrogen receptor-related γ (ERR γ) belongs to the estrogen-related receptor subclass of nuclear receptors and is highly expressed in tissues such as heart, kidney, brain, and skeletal muscles (see Heard et al., Molecular Endocrinology, 14(3): 382-386, 2000, which is hereby incorporated by reference in its entirety). ERRγ is a positive regulator of oxidative myofibers, mitochondrial biogenesis, and muscle angiogenesis, as well as exercise tolerance. Moreover, we showed using an ischemia model of muscle injury that ERRγ promotes muscle repair via metabolic/fiber type and angiogenic remodeling in the skeletal muscle. ERRγ (NR3B3) is a constitutively-active orphan nuclear receptor structurally homologous to estrogen receptors, composed of an N-terminal activation domain, a DNA binding domain comprised of two zinc fingers, and a C-terminal ligand binding domain. ERRγ (GenBank® Accession Nos. NM_001 134285.1, AY388461, AF058291.1 and NM_01 1935.2 disclose ERRγ nucleic acids, and GenBank® Accession Nos. NP_001127757.1, P62508.1, AAQ93381.1, and NP_036065.1 disclose ERRγ proteins each of which is hereby incorporated by reference as available on Jun. 18, 2014) is 76% identical to ERRβ and 63% identical to ERRα, and binds to estrogen response elements in genes to affect their transcription, but is not activated by estrogen.

Increase: To enhance the quality, amount, or strength of something. In one example, an agent increases the activity or expression of ERRγ, for example relative to an absence of the agent. In a particular example, an agent increases the activity or expression of ERRγ, related receptors ERRα or β, or ERR-regulated metabolic and angiogenic genes by at least 10%, at least 20%, at least 50%, or even at least 90%, including between 10% to 95%, 20% to 80%, 30% to 70%, 40% to 50%, such as 10%, 20%, 30%, 40%, 50%, 60%, 70%, 80%, 85%, 90%, 95%, 98%, or 100%. Such increases can be measured using the methods disclosed herein.

In a particular example, a therapy increases (also known as up-regulates) the expression of ERRγ, such as an increase of at least 10%, at least 20%, at least 50%, or even at least 90% in ERRγ expression, thereby treating/alleviating one or more signs or symptoms associated with muscular dystrophy. In some examples, an increase in expression refers to an increase in an ERRγ gene product. An ERRγ gene product can be RNA (such as mRNA, rRNA, tRNA, and structural RNA) or protein.

Gene upregulation includes any detectable increase in the production of an ERRγ gene product. In certain examples, production of an ERRγ gene product increases by at least 2-fold, for example at least 3-fold or at least 4-fold, as compared to a control (such an amount of gene expression in a normal cell). In one example, a control is a relative amount of ERRγ gene expression or protein expression in a biological sample taken from a subject who does not have muscular dystrophy, such as DMD, FCMD or MDC1A. Such increases can be measured using the methods disclosed herein. For example, “detecting or measuring expression of ERRγ” includes quantifying the amount of the gene, gene product or modulator thereof present in a sample. Quantification can be either numerical or relative. Detecting expression of the gene, gene product or modulators thereof can be achieved using any method known in the art or described herein, such as by measuring nucleic acids by PCR (such as RT-PCR) and proteins by ELISA. In primary embodiments, the change detected is an increase or decrease in expression as compared to a control, such as a reference value or a healthy control subject. In some examples, the detected increase or decrease is an increase or decrease of at least two-fold compared with the control or standard. Controls or standards for comparison to a sample, for the determination of differential expression, include samples believed to be normal (in that they are not altered for the desired characteristic, for example a sample from a subject who does not have muscular dystrophy, such as DMD, FCMD or MDC1A) as well as laboratory values (e.g., range of values), even though possibly arbitrarily set, keeping in mind that such values can vary from laboratory to laboratory.

Laboratory standards and values can be set based on a known or determined population value and can be supplied in the format of a graph or table that permits comparison of measured, experimentally determined values.

In other embodiments of the methods, the increase or decrease is of a diagnostically significant amount, which refers to a change of a sufficient magnitude to provide a statistical probability of the diagnosis.

The level of expression in either a qualitative or quantitative manner can detect nucleic acid or protein. Exemplary methods include microarray analysis, RT-PCR, Northern blot, Western blot, and mass spectrometry.

Improving muscular health: An improvement in muscular health compared with a preexisting state or compared with a state which would occur in the absence of treatment. For example, improving muscular health may include enhancing muscle regeneration, maintenance, or repair. Improving muscular health may also include prospectively treating a subject to prevent or reduce muscular damage or injury.

Inhibiting a disease or condition: A phrase referring to reducing the development of a disease or condition, for example, in a subject who is at risk for a disease or who has a particular disease. Particular methods of the present disclosure provide methods for inhibiting or reducing one or more signs or symptoms associated with muscular dystrophy, such DMD.

Ligand: Any molecule which specifically binds a protein, such as ERRγ, and includes, inter alia, antibodies that specifically bind to ERRγ. In alternative embodiments, the ligand is a protein or a small molecule.

Mimetic: A molecule (such as an organic chemical compound) that mimics the activity of an agent. Peptidomimetic and organomimetic embodiments are within the scope of this term, whereby the three-dimensional arrangement of the chemical constituents of such peptido- and organomimetics mimic the three-dimensional arrangement of the peptide backbone and component amino acid side chains in the peptide, resulting in such peptido- and organomimetics of the peptides having substantial specific activity. For computer modeling applications, a pharmacophore is an idealized, three-dimensional definition of the structural requirements for biological activity. Peptido- and organomimetics can be designed to fit each pharmacophore with computer modeling software (using computer assisted drug design or CADD). See Walters, “Computer-Assisted Modeling of Drugs”, in Klegerman & Groves, eds., 1993, Pharmaceutical Biotechnology, Interpharm Press: Buffalo Grove, Ill., pp. 165-174 and “Principles of Pharmacology” (ed. Munson, 1995), chapter 102 for a description of techniques used in computer assisted drug design.

Muscle: Any myoblast, myocyte, myofiber, myotube or other structure composed of muscle cells. Muscles or myocytes can be skeletal, smooth, or cardiac. Muscle may also refer to, in particular implementations of the present disclosure, cells or other materials capable of forming myocytes, such as stem cells and satellite cells.

Muscular dystrophy: A term used to refer to a group of genetic disorders that lead to progressive muscle weakness. Muscular dystrophy can result in skeletal muscle weakness and defects in skeletal muscle proteins, leading to a variety of impaired physiological functions. No satisfactory treatment of muscular dystrophy exists. Existing treatments typically focus on ameliorating the effects of the disease and improving the patient's quality of life, such as through physical therapy or through the provision of orthopedic devices.

Mutated genes associated with muscular dystrophy are responsible for encoding a number of proteins associated with the costameric protein network. Such proteins include laminin-2, collagen, dystroglycan, integrins, caveolin-3, ankyrin, dystrophin, α-dystrobrevin, vinculin, plectin, BPAG1b, muscle LIM protein, desmin, actinin-associated LIM protein, α-actin, titin, telethonin, cypher, myotilin, and the sarcoglycan/sarcospan complex.

The most common form of muscular dystrophy is DMD, affecting 1 in 3,500 live male births. DMD is an X-linked recessive disorder characterized by a mutation in the gene that codes for dystrophin. Dystrophin is a cytoskeletal protein about 430 kDa in size. This protein works to connect the cell's cytoskeleton and extracellular matrix. The loss of dystrophin in DMD patients leads to a loss of muscle fiber attachment at the extracellular matrix during contraction, which ultimately leads to progressive fiber damage, membrane leakage and a loss of muscle function. Most patients die before they reach the age of 30 due to respiratory or cardiac failure.

Beckers muscular dystrophy (also known as Benign pseudohypertrophic muscular dystrophy) is related to DMD in that both result from a mutation in the dystrophin gene, but in DMD no functional dystrophin is produced making DMD much more severe than BMD. BMD is an X-linked recessive inherited disorder characterized by slowly progressive muscle weakness of the legs and pelvis. BMD is a type of dystrophinopathy, which includes a spectrum of muscle diseases in which there is insufficient dystrophin produced in the muscle cells, results in instability in the structure of muscle cell membrane. This is caused by mutations in the dystrophin gene, which encodes the protein dystrophin. The pattern of symptom development of BMD is similar to DMD, but with a later, and much slower rate of progression.

Congenital muscular dystrophies are caused by gene mutations. FCMD and MDC1A are examples of congenital muscular dystrophies. MDC1A is a congenital muscular dystrophy due to a genetic mutation in the LAMA2 gene which results in lack of or complete loss of laminin-α2 protein. This loss of laminin-α2 leads to an absence of laminins-211/221. Laminins-211/221 are major components of the extracellular matrix and play a key role in muscle cell development. During muscle cell differentiation laminin binds to the αβ1 integrin. Without laminin-α2, muscle fibers are unable to adhere to the basement membrane and myotubes undergo apoptosis. Muscle regeneration also fails, leading to a loss of muscle repair and an increase in muscle fibrosis and inflammation. This chronic tissue injury is a major cause of morbidity and mortality in MDC1A.

Congenital Muscular Dystrophies (CMD) and Limb-Girdle muscular dystrophy (LGMD) are common forms of highly heterogeneous muscular dystrophies which can be distinguished by their age at onset. In CMD, onset of symptoms is at birth or within the first 6 months of life; in LGMD onset of symptoms is in late childhood, adolescence or even adult life. Inheritance in LGMD can be autosomal dominant (LGMD type 1) or autosomal recessive (LGMD type 2), CMD is recessively inherited. CMD and LGMD can overlap both clinically and genetically

MDC1A is a progressive muscle wasting disease that results in children being confined to a wheelchair, requiring ventilator assistance to breathe and premature death. Symptoms are detected at birth with poor muscle tone and “floppy” baby syndrome. DMD, BMD and LGMD are progressive muscle degenerative diseases usually diagnosed at 3-5 years of age when children show developmental delay including ability to walk and climb stairs. The disease is progressive and children are usually confined to a wheelchair in their teens and require ventilator assistance.

Fukuyama congenital muscular dystrophy (FCMD) is an inherited condition that predominantly affects the muscles, brain, and eyes. Congenital muscular dystrophies are a group of genetic conditions that cause muscle weakness and wasting (atrophy) beginning very early in life. Fukuyama congenital muscular dystrophy affects the skeletal muscles, which are muscles the body uses for movement. The first signs of the disorder appear in early infancy and include a weak cry, poor feeding, and weak muscle tone (hypotonia). Weakness of the facial muscles often leads to a distinctive facial appearance including droopy eyelids (ptosis) and an open mouth. In childhood, muscle weakness and joint deformities (contractures) restrict movement and interfere with the development of motor skills such as sitting, standing, and walking. Fukuyama congenital muscular dystrophy also impairs brain development. People with this condition have a brain abnormality called cobblestone lissencephaly, in which the surface of the brain develops a bumpy, irregular appearance (like that of cobblestones). These changes in the structure of the brain lead to significantly delayed development of speech and motor skills and moderate to severe intellectual disability. Social skills are less severely impaired. Most children with Fukuyama congenital muscular dystrophy are never able to stand or walk, although some can sit without support and slide across the floor in a seated position. More than half of all affected children also experience seizures. Other signs and symptoms of Fukuyama congenital muscular dystrophy include impaired vision, other eye abnormalities, and slowly progressive heart problems after age 10. As the disease progresses, affected people may develop swallowing difficulties that can lead to a bacterial lung infection called aspiration pneumonia. Because of the serious medical problems associated with Fukuyama congenital muscular dystrophy, most people with the disorder live only into late childhood or adolescence.

Fukuyama congenital muscular dystrophy is seen almost exclusively in Japan, where it is the second most common form of childhood muscular dystrophy (after Duchenne muscular dystrophy). Fukuyama congenital muscular dystrophy has an estimated incidence of 2 to 4 per 100,000 Japanese infants.

Fukuyama congenital muscular dystrophy is caused by mutations in the FKTN gene which encodes fukutin. The most common mutation in the FKTN gene reduces the amount of fukutin produced within cells. A shortage of fukutin likely prevents the normal modification of α-dystroglycan, which disrupts that protein's normal function. Without functional α-dystroglycan to stabilize muscle cells, muscle fibers become damaged as they repeatedly contract and relax with use. The damaged fibers weaken and die over time, leading to progressive weakness and atrophy of the skeletal muscles.

Defective α-dystroglycan also affects the migration of neurons during the early development of the brain. Instead of stopping when they reach their intended destinations, some neurons migrate past the surface of the brain into the fluid-filled space that surrounds it. Because Fukuyama congenital muscular dystrophy involves a malfunction of α-dystroglycan, this condition is described as a dystroglycanopathy.

Facioscapulohumeral muscular dystrophy (FHMD) is a form of muscular dystrophy associated with progressive muscle weakness and loss of muscle tissue. Unlike DMD and BMD which mainly affect the lower body, FSHD affects the upper body mainly the face, shoulder and upper arm muscles. However, it can affect muscles around the pelvis, hips, and lower leg. Symptoms for FSHD often do not appear until age 10-26, but it is not uncommon for symptoms to appear much later. In some cases, symptoms never develop. Symptoms are usually mild and very slowly become worse. Facial muscle weakness is common, and may include eyelid drooping, inability to whistle, decreased facial expression, depressed or angry facial expression, difficulty pronouncing words, shoulder muscle weakness (leading to deformities such as pronounced shoulder blades (scapular winging) and sloping shoulders), weakness of the lower, hearing loss and possible heart conditions.

Pharmaceutical agent: A chemical compound or composition capable of inducing a desired therapeutic or prophylactic effect when properly administered to a subject or a cell. “Incubating” includes a sufficient amount of time for a drug to interact with a cell. “Contacting” includes incubating a drug in solid or in liquid form with a cell. A “therapeutically effective amount” is a quantity of a chemical composition sufficient to achieve a desired effect in a subject being treated. For instance, this can be the amount necessary to prevent, reduce or inhibit one or more signs or symptoms associated with muscular dystrophy.

Pharmaceutically acceptable carriers: The pharmaceutically acceptable carriers of use are conventional. Remington's Pharmaceutical Sciences, by E. W. Martin, Mack Publishing Co., Easton, Pa., 19th Edition, 1995, describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives (such as natural and/or non-natural preservatives), and pH buffering agents and the like, for example sodium acetate or sorbitan monolaurate.

Polypeptide: Any chain of amino acids, regardless of length or post-translational modification (such as glycosylation or phosphorylation). “Polypeptide” applies to amino acid polymers to naturally occurring amino acid polymers and non-naturally occurring amino acid polymer as well as in which one or more amino acid residue is a non-natural amino acid, for example a artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used interchangeably herein to refer to a polymer of amino acid residues.

Purified: The term purified does not require absolute purity; rather, it is intended as a relative term. Thus, for example, a purified protein is one in which the protein is more enriched than the protein is in its natural environment within a cell. Preferably, a preparation is purified such that the protein represents at least 50% of the protein content of the preparation.

The polypeptides or antibodies disclosed herein can be purified by any of the means known in the art. See for example Guide to Protein Purification, ed. Deutscher, Meth. Enzymol. 185, Academic Press, San Diego, 1990; and Scopes, Protein Purification: Principles and Practice, Springer Verlag, New York, 1982. Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Regeneration: A term which refers to the repair of cells or tissue, such as muscle cells or tissue (or organs) which includes muscle cells, following injury or damage to at least partially restore the muscle or tissue to a condition similar to which the cells or tissue existed before the injury or damage occurred. Regeneration also refers to facilitating repair of cells or tissue in a subject having a disease affecting such cells or tissue to eliminate or ameliorate the effects of the disease. In more specific examples, regeneration places the cells or tissue in the same condition or an improved physiological condition as before the injury or damage occurred or the condition which would exist in the absence of disease.

Repair of cells or tissue (such as muscle cells or tissue (or organs) includes muscle cells): a physiological process of healing damage to the cells or tissue following damage or other trauma.

Specific Binding Agent: An agent that binds substantially or preferentially only to a defined target such as a protein, enzyme, polysaccharide, oligonucleotide, DNA, RNA, recombinant vector or a small molecule. A protein-specific binding agent binds substantially only the defined protein, or to a specific region within the protein. For example, a “specific binding agent” includes antibodies and other agents that bind substantially to a specified polypeptide, such as ERRγ. Antibodies can be monoclonal or polyclonal antibodies that are specific for the polypeptide, as well as immunologically effective portions (“fragments”) thereof. The determination that a particular agent binds substantially only to a specific polypeptide may readily be made by using or adapting routine procedures. One suitable in vitro assay makes use of the Western blotting procedure (described in many standard texts, including Harlow and Lane, Using Antibodies: A Laboratory Manual, CSHL, New York, 1999). In an example, a “specific binding agent” is capable of binding to ERRγ thereby preventing, reducing and/or inhibiting one or signs or symptoms associated with muscular dystrophy.

Signs or symptoms: Any subjective evidence of disease or of a subject's condition, e.g., such evidence as perceived by the subject; a noticeable change in a subject's condition indicative of some bodily or mental state. A “sign” is any abnormality indicative of disease, discoverable on examination or assessment of a subject. A sign is generally an objective indication of disease. Signs include, but are not limited to any measurable parameters such as tests for detecting muscular dystrophy, including measuring creatine kinase levels, electromyography (to determine if weakness is caused by destruction of muscle tissue rather than by damage to nerves) or immunohistochemistry/immunoblotting/immunoassay (e.g., ELISA) to measure muscular dystrophy-associated molecules, such as ERRγ or other molecules shown in the examples below to be associated with muscular dystrophy. In one example, reducing or inhibiting one or more symptoms or signs associated with muscular dystrophy, includes increasing the activity or expression of ERRγ by a desired amount, for example by at least 10%, at least 20%, at least 50%, at least 60%, at least 70%, at least 80%, at least 90%, at least 95%, at least 98%, or even at least 100%, as compared to the activity and/or expression in the absence of the treatment. Symptoms of muscular dystrophy include, but are not limited to, muscle weakness and loss, difficulty running, difficulty hopping, difficulty jumping, difficulty walking, difficulty breathing, fatigue, skeletal deformities, muscle deformities (contractions of heels; pseudohypertrophy of calf muscles), heart disease (such as dilated cardiomyopathy), elevated creatine phosphokinase (CK) levels in blood or combinations thereof.

Subject: Living multi-cellular vertebrate organisms, a category that includes human and non-human mammals. In an example, a subject is a human. In an additional example, a subject is selected that is in need of preventing, reducing and/or inhibiting one or more signs or symptoms associated with muscular dystrophy.

Test Agent: Any substance, including, but not limited to, a protein (such as an antibody), a nucleic acid molecule (such as a siRNA), an organic compound, an inorganic compound, a small molecule, a peptide or any other molecule of interest.

Treating a disease: A therapeutic intervention that ameliorates a sign or symptom of a disease or pathological condition related to a muscular dystrophy, such as a sign or symptom of muscular dystrophy. Treatment can induce remission or cure of a condition or slow progression, for example, in some instances can include inhibiting the full development of a disease, for example preventing development of a muscular dystrophy. Prevention of a disease does not require a total absence of disease. For example, a decrease of at least 50% can be sufficient.

Treating a disease can be a reduction in severity of some or all clinical symptoms of the disease or condition, a reduction in the number of relapses of the disease or condition, an improvement in the overall health or well-being of the subject, by other parameters well known in the art that are specific to the particular disease or condition, and combinations of such factors.

Under conditions sufficient for: A phrase that is used to describe any environment that permits the desired activity. In one example, includes administering a disclosed agent to a subject sufficient to allow the desired activity. In particular examples, the desired activity is increasing the expression or activity of ERRγ.

### III. Methods of Use

Disclosed herein are methods of improving muscular health, such as enhancing muscle regeneration, maintenance, or repair. In some examples, the method includes administering to the subject an effective amount of an agent capable of increasing activity or expression of ERRγ, related receptors ERRα or β, or ERR-regulated metabolic and angiogenic genes, thereby enhancing muscle regeneration, maintenance, or repair. In some examples, these methods include contacting a cell with an effective amount of an ERRγ modulatory agent and increases ERRγ expression or activity in the treated cell relative to ERRγ expression in an untreated cell, thereby enhancing ERRγ expression or activity. In some examples, the cell is a muscle cell, such as a skeletal muscle cell. In some examples, the muscle cell is present in a mammal, and wherein contacting the cell with an agent comprises administering the agent to the mammal. In some examples, the disclosed ERRγ modulatory agents can increase the expression of nucleic acid sequences (such as DNA, cDNA, or mRNAs) and proteins of ERRγ. An increase in the expression or activity does not need to be 100% for the agent to be effective. For example, an agent can increase the expression or biological activity by a desired amount, for example by at least 10%, for example at least 20%, at least 50%, at least 60%, at least 70%, at least 80%, at least 90%, at least 95%, at least 98%, or even at least 100%, including about 15% to about 98%, about 30% to about 95%, about 40% to about 80%, about 50% to about 70%, including about 20%, about 30%, about 40%, about 50%, about 60%, about 70%, about 80%, about 90%, about 95%, about 98% or about 100%, as compared to activity or expression in a control. Methods of assessing ERRγ expression and activity are described herein and in particular, the Examples below. In other examples, methods of improving muscular health include administering to the subject an effective amount of an agent capable of inhibiting activity or expression of ERRγ, related receptors ERRα or β, or ERR-regulated metabolic and angiogenic genes.

In some examples, the disclosed methods are utilized to treat, prevent or inhibit a muscular disease. In some embodiments, the muscular disease is a muscular dystrophy. In some specific embodiments, the muscular dystrophy is Duchenne muscular dystrophy, Becker muscular dystrophy, limb-girdle muscular dystrophy, congenital muscular dystrophy, facioscapulohumeral muscular dystrophy, myotonic muscular dystrophy, oculopharyngeal muscular dystrophy, distal muscular dystrophy, or Emery-Dreifuss muscular dystrophy. In some embodiments, the muscular dystrophy is Duchenne muscular dystrophy. In some embodiments, the disease is a demyelinating disease. In some further embodiments, the demyelinating disease is multiple sclerosis, Charcot-Marie-Tooth disease, Pelizaeus-Merzbacher disease, encephalomyelitis, neuromyelitis optica, adrenoleukodystrophy, and/or Guillian-Barre syndrome. In some examples, the methods are utilized to treat a subject with one or more signs or symptoms of muscular disease such as a muscular dystrophy, such as, but not limited to DMD. In some examples, the disclosed methods further include selecting a subject in need of enhancing muscle regeneration, maintenance, or repair. For example, the method includes diagnosing the subject as having a condition treatable by administering an agent capable of increasing activity or expression of ERRγ, related receptors ERRα or β, or ERR-regulated metabolic and angiogenic genes. In one example, the subject is diagnosed as suffering from muscular dystrophy, such as a congenital muscular dystrophy, DMD, or Limb-girdle muscular dystrophy, for example by having a decreased expression level of one or more of ERRγ, related receptors ERRα or β, or ERR-regulated metabolic and angiogenic genes. In further instances the condition is characterized by the failure of a subject, or the reduced ability of the subject, to express one or more proteins associated with the formation or maintenance of the extracellular matrix, such as impaired or non-production of a laminin, an integrin, dystrophin, utrophin, or dystroglycan.

In some examples, the present disclosure also provides a method for increasing muscle regeneration in a subject. For example, geriatric subjects, subjects suffering from muscle disorders, and subjects suffering from muscle injury, including activity induced muscle injury, such as injury caused by exercise, may benefit from this embodiment.

In some examples of the disclosed method, an agent capable of increasing ERRγ is administered in a preventative manner, such as to prevent or reduce muscular damage or injury (such as activity or exercise induced injury). For example, geriatric subjects, subjects prone to muscle damage, or subjects at risk for muscular injury, such as athletes, may be treated in order to eliminate or ameliorate muscular damage, injury, or disease.

In further embodiments, the method of the present disclosure includes administering one or more additional pharmacological substances, such as a therapeutic agent. In some aspects, the additional therapeutic agent enhances the therapeutic effect of the agent capable of increasing ERRγ, related receptors ERRα or β, or ERR-regulated metabolic and angiogenic genes activity or expression. In further aspects, the therapeutic agent provides independent therapeutic benefit for the condition being treated. In various examples, the additional therapeutic agent is a component of the extracellular matrix, such as an integrin, dystrophin, dystroglycan, utrophin, or a growth factor. In further examples, the therapeutic agent reduces or enhances expression of a substance that enhances the formation or maintenance of the extracellular matrix.

In some examples, the ERRγ, related receptors ERRα or β, or ERR-regulated metabolic and angiogenic genes agent is applied to a particular area of the subject to be treated, such as a muscle. In further examples, the specific agent is administered such that it is distributed to multiple areas of the subject, such as systemic administration or regional administration. It is contemplated that any route of administered suitable for the method can be utilized, such as topically, parenterally (such as intravenously or intraperitoneally), or orally.

Although the disclosed methods generally have been described with respect to muscle regeneration, the disclosed methods also may be used to enhance repair or maintenance, or prevent damage to, other tissues and organs. For example, the methods of the present disclosure can be used to treat symptoms of muscular dystrophy stemming from effects to cells or tissue other than skeletal muscle, such as impaired or altered brain function, smooth muscles, or cardiac muscles.

In some embodiments, the disclosed methods are utilized to treat subjects with DMD or other types of muscular dystrophy. In other embodiments, the disclosed methods are utilized to treat subjects with other types of muscular dystrophy in addition to DMD, such as those defined herein or known to one of skill in the art.

In an example, muscular dystrophy, such as DMD, can be reduced or inhibited by contacting a cell with an effective amount of an agonist, such as an agent capable of increasing ERRγ, ERRα or ERRβ, or ERR-regulated metabolic and angiogenic genes or protein activity or expression (e.g., an antibody, a peptide agonist, a peptidomimetic agonist, a proteomimetic agonist, or a small molecule agonist). In some examples, the agent specifically increases the activation or function of ERRγ, ERRα, ERRβ, or ERR-regulated metabolic and angiogenic genes or protein activity or expression and thereby reduces or inhibits one or more signs or symptoms associated with muscular dystrophy, such as DMD.

The signs and symptoms of muscular dystrophy do not need to be completely eliminated for the composition to be effective. For example, a composition can reduce the symptoms by a desired amount, for example by at least 10%, at least 20%, at least 50%, at least 60%, at least 70%, at least 80%, at least 90%, at least 95%, at least 98%, or even at least 100% (elimination of detectable muscular dystrophy), including about 15% to about 98%, about 30% to about 95%, about 40% to about 80%, about 50% to about 70%, including about 20%, about 30%, about 40%, about 50%, about 60%, about 70%, about 80%, about 90%, about 95%, about 98% or about 100%, as compared to the signs and symptoms being displayed in the absence of the composition. In some examples, the cell is also contacted with an effective amount of an additional agent, such as an additional agent capable of modulating one or more signs or symptoms associated with muscular dystrophy. The cell can be in vivo or in vitro.

### Exemplary Agents

Exemplary agents for any of the disclosed methods are those that modulate, such as increase or decrease expression and/or activity of ERRγ, related receptors ERRα or β, and/or ERR-regulated metabolic and angiogenic genes. Examples of ERRγ-specific modulators include antibodies, ligands, recombinant proteins, peptide mimetics, soluble receptor fragments and siRNAs. In some examples, an activating agent is an ERRγ ligand that is capable of increasing the expression and/or activity of ERRγ. In some examples, a therapeutically effective activating agent is one that when administered in therapeutically effective amounts induce the desired response (e.g., enhancement of muscle regeneration, maintenance, or repair and/or treating muscular dystrophy, such as DMD). In one example, activating agents bind with higher affinity to a molecule of interest, such as ERRγ, than to other molecules, such as ERRα or β. For example, a therapeutic agent can be an ERRγ-specific agonist that binds with high affinity to ERRγ, but does not substantially bind to other ERRs. For example, an ERRγ specific agonist binds to ERRγ with a binding affinity in the range of 0.1 to 10 μM (such as 0.1 to 1 μM, 1 to 10 μM, 5 to 10 μM, or 10 to 20 μM) and increases the activity of such, thereby generating the desired therapeutic effect (e.g., enhancing muscle regeneration, maintenance, or repair and/or treating muscular dystrophy or other muscle disorder). In some examples, an ERRγ specific agonist is GW4064 or a GW4064 analog (see WO/2004/046068 which is hereby incorporated by reference in its entirety), bisphenol A (BPA), GSK 4716 or an analog thereof. The chemical name for GW4064 is benzoic acid, 3-[2-[2-chloro-4-[[3-(2,6-dichlorophenyl)-5-(1-methylethyl)-4-isoxazolyl]methoxy]phenyl]ethenyl]- and it has a molecular weight of 542.84. GW4064 is commercially available, such as from Sigma-Aldrich, Tocris Bioscience, and Selleck Chemicals). In some examples, an activating agent is one or more of the phenolic acyl hydrazones selective agonists for ERRβ and ERRγ disclosed in Journal of Medicinal Chemistry (48 3107, 2006; which is hereby incorporated by reference in its entirety). In some examples, an activating agent is a phenolic acyl hydrazones shown to act as selective agonists for ERRγ (Zuercher et al., Identification and structure-activity relationship of phenolic acyl hydrazones as selective agonists for the estrogen-related orphan nuclear receptors ERRbeta and ERRgamma. 2005, J. Med. Chem. 48:3107-9, which is hereby incorporated by reference in its entirety), including, but not limited to, phenolic acyl hydrazone GSK4716 or GSK9089. GSK 4716 is a selective agonist with estrogen-related receptors ERRβ and ERRγ and it is widely available commercially (see, for example, Tocris Bioscience, Santa Cruz Biotechnology, Inc., Sigma-Aldrich. Abeam and Fisher Scientific). In some examples, an activating agent is DY131 (Tocris Bioscience, Santa Cruz Biotechnology, R&D Systems). In some examples, an activating agent is a flavone (6,3′,4′-trihydroxyflavone), or an isoflavone (genistein, daidzein and biochanin A) phytoestrogens disclosed in (Suetsugi et al., Flavone and isoflavone phytoestrogens are agonists of estrogen-related receptors. Mol Cancer Res. 2003, 1:981-91, which is hereby incorporated by reference in its entirety).

In some examples, an inhibiting agent is an ERRγ ligand that is capable of reducing and/or inhibiting the expression and/or activity of ERRγ. In some examples, a therapeutically effective inhibiting agent is one that when administered in therapeutically effective amounts induce the desired response (e.g., reducing muscle regeneration, maintenance, or repair). In one example, inhibiting agents bind with higher affinity to a molecule of interest, such as ERRγ, than to other molecules, such as ERRα or β. For example, a therapeutic agent can be an ERRγ-specific antagonist that binds with high affinity to ERRγ, but does not substantially bind to other ERRs. For example, an ERRγ specific antagonist binds to ERRγ with a binding affinity in the range of 0.1 to 10 μM (such as 0.1 to 1 μM, 1 to 10 μM, 5 to 10 μM, or 10 to 20 μM) and decreases the activity of such, thereby generating the desired therapeutic effect (e.g., reducing muscle regeneration, maintenance, or repair). In some examples, an ERRγ antagonist is 4-hydroxytamoxifen or diethylstilbestrol (DES). In some examples, an antagonist is an inverse agonist thiadiazolopyrimidinone Ia or a derivative XCT790 which interfere with PGC-1/ERRα dependent signaling (Busch et al., Identification of a selective inverse agonist for the orphan nuclear receptor estrogen-related receptor alpha. J Med Chem. 2004, 47:5593-6. and Willy et al., Regulation of PPARgamma coactivator 1alpha (PGC-1alpha) signaling by an estrogen-related receptor alpha (ERRalpha) ligand. Proc Natl Acad Sci USA. 2004, 101:8912-7 each of which is hereby incorporated by reference in its entirety). In some examples, an ERRα antagonist is Octochlorocamphene (Yang C (1999) Two organochlorine pesticides, toxaphene and chlordane, are antagonists for estrogen-related receptor alpha-1 orphan receptor. Cancer Res 59, 4519-24, which is hereby incorporated by reference in its entirety) and an ERRα inverse agonist is XCT790 (Busch B B, Stevens W C, Martin R, Ordentlich P, Zhou S, Sapp D W, Horlick R A and Mohan R (2004) Identification of a selective inverse agonist for the orphan nuclear receptor estrogen-related receptor alpha. J Med Chem 47, 5593-6, which is hereby incorporated by reference in its entirety).

Administration

The disclosed agents or other therapeutic substance are in general administered topically, nasally, intravenously, orally, intracranially, intramuscularly, parenterally or as implants, but even rectal or vaginal use is possible in principle. The disclosed EERγ modulatory agents also may be administered to a subject using a combination of these techniques.

In a particular example, an ERRγ modulator is administered intravenously to a mammalian subject, such as a human. The therapeutically effective amount of the agents administered can vary depending upon the desired effects and the subject to be treated. In one example, the method includes daily administration of at least 1 μg of an ERRγ modulatory agent to the subject (such as a human subject). For example, a human can be administered at least 1 μg or at least 1000 mg of the agent daily, such as 10 μg to 100 μg daily, 100 μg to 1 mg daily, 100 μg to 1000 mg for example 100 μg daily, 1 mg daily, 10 mg daily, 100 mg daily, or 1000 mg. In an example, the subject is administered at least 1 μg (such as 1-100 μg) intravenously of the ERRγ modulatory agent. In one example, the subject is administered at least 1 mg intramuscularly (for example in an extremity) of such composition. The dosage can be administered in divided doses (such as 2, 3, or 4 divided doses per day), or in a single dosage daily. In a specific example, the subject is administered at least 0.15 mg per kg of body weight of the agent approximately every four weeks for at least 6 months. For example, 0.15 mg/kg, 0.5 mg/kg, 2 mg/kg, 3 mg/kg, 5 mg/kg or 6 mg/kg is administered, such as via intravenous or subcutaneous injections, every 28 days for 6 months.

In particular examples, the subject is administered a ERRγ modulatory agent on a multiple daily dosing schedule, such as at least two consecutive days, 10 consecutive days, and so forth, for example for a period of weeks, months, or years. In one example, the subject is administered the ERRγ modulatory agent daily for a period of at least 30 days, such as at least 2 months, at least 4 months, at least 6 months, at least 12 months, at least 24 months, or at least 36 months.

In specific examples, the agent for administration can include a solution of a disclosed ERRγ modulatory agent dissolved in a pharmaceutically acceptable carrier, such as an aqueous carrier. A variety of aqueous carriers can be used, for example, buffered saline and the like. These solutions are sterile and generally free of undesirable matter. These agents may be sterilized by conventional, well known sterilization techniques. The agents may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antibody or peptide in these formulations can vary widely, and will be selected primarily based on fluid volumes, viscosities, body weight and the like in accordance with the particular mode of administration selected and the subject's needs.

A typical pharmaceutical composition for intravenous administration includes about 0.1 to 10 mg of the disclosed ERRγ modulatory agent per subject per day. Dosages from 0.1 up to about 100 mg per subject per day may be used. Actual methods for preparing administrable compositions are described in more detail in such publications as Remington's Pharmaceutical Science, 19th ed., Mack Publishing Company, Easton, Pa. (1995).

The disclosed agents may be provided in lyophilized form and rehydrated with sterile water before administration, although they are also provided in sterile solutions of known concentration. The agent solution is then added to an infusion bag containing 0.9% Sodium Chloride, USP, and typically administered at a dosage of from 0.5 to 15 mg/kg of body weight. The disclosed agents can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30 minute period if the previous dose was well tolerated.

In some examples, a therapeutically effective amount of a nucleic acid molecule is administered to a subject, such as an siRNA molecule. Nucleic acid molecules, such as siRNA specific for ERRγ can be administered to cells by a variety of methods known to those of skill in the art, including, but not restricted to, encapsulation in liposomes, by iontophoresis, or by incorporation into other delivery vehicles, such as hydrogels, cyclodextrins, biodegradable nanocapsules, and bioadhesive microspheres, or by proteinaceous vectors (see, for example, O'Hare and Normand, International PCT Publication No. WO 00/53722). Alternatively, the nucleic acid/vehicle combination can be locally delivered by direct injection or by use of an infusion pump. Direct injection of the nucleic acid molecules of the disclosure, whether subcutaneous, intramuscular, or intradermal, can take place using standard needle and syringe methodologies, or by needle-free technologies such as those described by Barry et al., International PCT Publication No. WO 99/31262. Other delivery routes include, but are not limited to, oral delivery (such as in tablet or pill form), intrathecal or intraperitoneal delivery. More detailed descriptions of nucleic acid delivery and administration are provided in Sullivan et al., PCT WO 94/02595, Draper et al., PCT WO93/23569, Beigelman et al., PCT WO99/05094 and Klimuk et al., PCT WO99/04819.

The disclosed agents including one or more ERRγ modulatory agents, can further include one or more biologically active or inactive compounds (or both), and conventional non-toxic pharmaceutically acceptable carriers, respectively. Examples of such biologically inactive compounds include, but are not limited to: carriers, thickeners, diluents, buffers, preservatives, and carriers. The pharmaceutically acceptable carriers useful for these formulations are conventional (see Remington's Pharmaceutical Sciences, by E. W. Martin, Mack Publishing Co., Easton, Pa., 19th Edition (1995)). In general, the nature of the carrier will depend on the particular mode of administration being employed. For instance, parenteral formulations can include injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can include minor amounts of non-toxic auxiliary substances, such as wetting or emulsifying agents, preservatives, and pH buffering agents and the like, for example sodium acetate or sorbitan monolaurate.

Controlled release parenteral formulations can be made as implants, oily injections, or as particulate systems. For a broad overview of protein delivery systems see, Banga, Therapeutic Peptides and Proteins: Formulation, Processing, and Delivery Systems, Technomic Publishing Company, Inc., Lancaster, Pa., (1995) incorporated herein by reference. Particulate systems include microspheres, microparticles, microcapsules, nanocapsules, nanospheres, and nanoparticles. Microcapsules contain the therapeutic protein, such as a cytotoxin or a drug, as a central core. In microspheres the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, Colloidal Drug Delivery Systems, ed., Marcel Dekker, Inc., New York, N.Y., pp. 219-342 (1994); and Tice & Tabibi, Treatise on Controlled Drug Delivery, ed., Marcel Dekker, Inc. New York, N.Y., pp. 315-339, (1992) both of which are incorporated herein by reference.

Polymers can be used for ion-controlled release of the agents disclosed herein. Various degradable and nondegradable polymeric matrices for use in controlled drug delivery are known in the art (Langer, Accounts Chem. Res., 26: 537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res., 9: 425-434, 1992; and Pec et al., J. Parent. Sci. Tech., 44(2): 58-65, 1990). Alternatively, hydroxyapatite has been used as a microcarrier for controlled release of proteins (Ijntema et al., Int. J. Pharm., 112: 215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa. (1993)). Numerous additional systems for controlled delivery of therapeutic proteins are known (see U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342 and U.S. Pat. No. 5,534,496).

ii. Desired Response

One or more disclosed ERRγ modulatory agents and/or additional therapeutic agents are administered by a specific route and/or concentration to generate a desired response. In some examples, a desired response refers to an amount effective for lessening, ameliorating, eliminating, preventing, or inhibiting at least one symptom of a disease, disorder, or condition treated and may be empirically determined. In various embodiments of the present disclosure, a desired response is muscle regeneration, reductions or prevention of muscle degeneration, promotion of muscle maintenance, reduction or prevention of muscle injury or damage, reduction or prevention in one more signs or symptoms associated with muscular dystrophy.

In particular, indicators of muscular health, such as muscle cell regeneration, maintenance, or repair, can be assessed through various means, including monitoring markers of muscle regeneration, such as transcription factors such as Pax7, Pax3, MyoD, MRF4, and myogenin. For example, increased expression of such markers can indicate that muscle regeneration is occurring or has recently occurred. Markers of muscle regeneration, such as expression of embryonic myosin heavy chain (eMyHC), can also be used to gauge the extent of muscle regeneration, maintenance, or repair. For example, the presence of eMyHC can indicate that muscle regeneration has recently occurred in a subject.

Muscle cell regeneration, maintenance, or repair can also be monitored by determining the girth, or mean cross sectional area, of muscle cells or density of muscle fibers. Additional indicators of muscle condition include muscle weight and muscle protein content. Mitotic index (such as by measuring BrdU incorporation) and myogenesis can also be used to evaluate the extent of muscle regeneration.

In particular examples, the improvement in muscle condition, such as regeneration, compared with a control is at least about 10%, such as at least about 30%, or at least about 50% or more, including an at least 15%, at least 20%, at least 25%, at least 30%, at least 40%, at least 45%, at least 50%, at least 60%, at least 70%, at least 80%, at least 90%, including a 10% to 90% decrease, 20% to 80% increase, 30% to 70% increase or a 40% to 60% increase (e.g., a 10%, 20%, 30%, 40%, 50%, 60%, 70%, 80%, 90%, 95%, 100%, 200% or more increase).

### Additional Treatments

In particular examples, prior to, during, or following administration of an effective amount of an agent that reduces or inhibits one or more signs or symptoms associated with muscular dystrophy, the subject can receive one or more other therapies. In one example, the subject receives one or more treatments prior to administration of a disclosed ERRγ-modulatory agent. In some examples, a source of muscle cells can be added to aid in muscle regeneration and repair. In some aspects of the present disclosure, satellite cells are administered to a subject in combination with ERRγ therapy. U.S. Patent Publication 2006/0014287, incorporated by reference herein to the extent not inconsistent with the present disclosure, provides methods of enriching a collection of cells in myogenic cells and administering those cells to a subject. In further aspects, stem cells, such as adipose-derived stem cells, are administered to the subject. Suitable methods of preparing and administering adipose-derived stem cells are disclosed in U.S. Patent Publication 2007/0025972, incorporated by reference herein to the extent not inconsistent with the present disclosure. Additional cellular materials, such as fibroblasts, can also be administered, in some examples.

### Additional Therapeutic Agents

Additional therapeutic agents include agents which enhance the effect of the disclosed modulatory agents, such as a component of the extracellular matrix or a growth factor. In some examples, the additional substance can include aggrecan, angiostatin, cadherins, collagens (including collagen I, collagen III, or collagen IV), decorin, elastin, enactin, endostatin, fibrin, fibronectin, osteopontin, tenascin, thrombospondin, vitronectin, and combinations thereof. Biglycans, glycosaminoglycans (such as heparin or heparan sulfate), glycoproteins (such as dystroglycan), proteoglycans (such as decorin or biglycan), and combinations thereof can also be administered.

In some examples, growth stimulants such as cytokines, polypeptides, and growth factors such as brain-derived neurotrophic factor (BDNF), CNF (ciliary neurotrophic factor), EGF (epidermal growth factor), FGF (fibroblast growth factor), glial growth factor (GGF), glial maturation factor (GMF) glial-derived neurotrophic factor (GDNF), hepatocyte growth factor (HGF), insulin, insulin-like growth factors, kerotinocyte growth factor (KGF), nerve growth factor (NGF), neurotropin-3 and -4, PDGF (platelet-derived growth factor), vascular endothelial growth factor (VEGF), and combinations thereof may be administered with one of the disclosed methods.

### IV. Identifying ERRγ Agents

Methods are provided herein for identifying agents to activate or inhibit ERRγ, such as agents that increase the activity of ERRγ and can be used to enhance muscle regeneration, maintenance, or repair and/or treat one or more signs or symptoms associated with muscular dystrophy. In one example, the method includes contacting at least one cell with one or more test agents wherein the cell expresses at least ERRγ. The method can also include detecting the affinity, such as a decrease or increase in the binding of a synthetic ligand of ERRγ relative to a control, such as the binding of one of the synthetic ligands in the absence of the one or more test agents. In several examples, an increase in the binding of the synthetic ligand of ERRγ relative to a control indicates that the one or more test agents is of use to enhance muscle regeneration, maintenance, or repair and/or prevent and/or treat muscular dystrophy.

In one example, determining whether there is an increase in binding of one or more synthetic ligands of ERRγ is by use of an in vitro assay. For example, an in vitro assay can be employed to compare activity of one or more such synthetic ligands in the presence and absence of the one or more test agents. Various types of in vitro assays may be employed to identify agents to enhance muscle regeneration, maintenance, or repair and/or prevent, inhibit or treat muscular dystrophy including, but not limited to, transfection and transactivation assay systems. In one example, ERRγ ligands are identified by transfecting cells with full length mouse ERRγ (cloned into a modified pcDNA3.1-vector) (INVITROGEN®) using Lipofectamine 2000 (INVITROGEN®) following manufacturer's instructions. Each transfection contains empty vector (control) or ERRγ wild-type (WT) or mutant expression plasmid, pGL3 ERRE X3 promoter vector (Evans Laboratory). In some examples, a GAL4 fusion of the ERRγ ligand binding domain (LBD) and MH2004 Luc (contains 4 elements of a Gal4 DBD binding sites) is used. Luciferase activities can be measured, such as by use of a Perkin Elmer Envision Luminometer using the Dual-Luciferase reporter assay system (Promega).

In additional examples, commercially available assays are used to screen for ERRγ ligands. For example, a LanthaScreen Estrogen Related Receptor gamma TR-FRET co-activator assay (INVITROGEN®) is used to identify ERRγ ligands.

The foregoing disclosure is further explained by the following non-limiting examples.

### EXAMPLES

### Example 1

### Material and Methods

This Example provides the Materials and Methods used for the studies described in Examples 2-9.

Animal Husbandry.

Generation of the muscle-specific ERRγ transgenic mice using human α-skeletal actin promoter. Male ERRγ transgenic mice were bred to homozygous female mdx mice (Jackson Laboratories, Bar Harbor, Me., USA) and maintained in the vivarium at the Brown Foundation Institute of Molecular Medicine (University of Texas Medical School, Houston, Tex., USA). Male offspring were genotyped and categorized in mdx and mdx-ERRγ overexpresser (mdx-tg) cohorts. Age- and sex-matched C57B1/6J mice were primarily used as wild-type controls. (Where indicated, age- and sex-matched C57B1/10J mice were used as additional wild-type controls.) Mice were housed under standard environmental conditions (20-22° C., 12:12-h light-dark cycle) and provided tap water ad libitum. Six- to 8-wk-old mice were used in all the studies.

Endurance Exercise.

Mdx and mdx-tg mice were subjected to endurance exercise tests. To detect maximum exercise tolerance, mice were subjected to swimming in groups until they reached exhaustion, which was defined by the inability to keep their nose above the water surface for breathing. Note that the control C57B1/6J mice were allowed to swim for 90 min, and they were removed from the water without any signs of exhaustion.

Serum Creatine Kinase (CK).

Serum CK was assayed in blood collected form rodent tails while the animals were under anesthesia induced by intraperitoneal injections of ketamine (100 mg/kg) and xylazine (10 mg/kg). Blood was allowed to clot for 1 h and centrifuged at 13,000 rpm for 20 min. Serum was collected and stored at −80° C., and samples were assayed within 1 week from collection using a CK reagent set from Pointe Scientific, Inc. (Canton, Mich., USA) according to the manufacturer's protocol. For determination of CK levels postexercise, mice were subjected to swimming in shallow water for 30 min/d for 4 consecutive days. Tail blood was collected 1 h after the end of the last bout on day 4.

Hypoxyprobe.

Postexercise tissue hypoxia was detected by immunofluorescence in Tibialis anterior (TA) and gastrocnemius muscle cryosections, obtained from mdx and mdx-tg mice, using a hypoxyprobe (pimonidazole hydrochloride) kit (cat. no. HP7-x; Hypoxyprobe, Inc., Burlington, Mass., USA) according to the manufacturer's instructions. Hypoxyprobe (60 mg/kg body weight) was administered by intraperitoneal injections immediately before the 30 min exercise bout, and tissues were collected 45 min postinjection.

Muscle Damage.

Muscle damage was detected using Evans blue dye (EBD) exclusion test. EBD solution (1% w/v in saline) was prepared and sterilized by passage through a 0.22-μm filter. It was intraperitoneally administered 24 h before specimen collection. Because EBD is impenetrable across intact membrane (or viable cells), it is excluded from intact skeletal muscle myofibers, but it selectively stains damaged myofibers. The EBD staining was visualized in muscle cryosections as fluorescence using excitation wavelength between 470 and 540 nm and an emission wavelength at 680 nm. Skeletal muscle damage in TA and gastrocnemius was evaluated as the function of EBD staining in the cryosections.

Fluorescence Microangiography.

Fluorescence microangiography was performed by subjecting anesthetized mice to intracardiac perfusion with 10 ml of PBS followed by a fluorescent microsphere (0.1 μM) suspension. Next, the mice were euthanized, and tissues were collected and appropriately frozen. Transverse cryosections of the TA and gastrocnemius muscles were processed and subjected to fluorescent microscopy to image skeletal muscle vasculature.

Microfil Perfusion and Imaging.

Whole-mount vascular mapping of the TA vasculature was performed using a 12% (w/v) microfil pigment solution of gouache in 4% PFA. The microfil solution was administered by intracardiac route in anesthetized mice, and the TA muscles were dissected, followed by serial dehydration in alcohol. Next, the tissues were incubated in fresh transparency solution that consisted of 1:1 benzylbenzoate and benzylalcohol until tissues became transparent. After the muscles were processed, whole-mount tissue images were obtained on an inverted microscope.

Laser Doppler Blood Flow Measurement.

Blood flow was measured with a deep tissue laser Doppler probe (Laserflo BPM2; Vasamedics, St. Paul, Minn., USA). Multiple measurements were made along the length of the TA and gastrocnemius and averaged for each muscle.

Muscle Histology.

Hind-limb muscles, such as TA and gastrocnemius, were collected at various time points pre- or postexercise, weighed, immersed in liquid nitrogen-chilled melting isopentane, and stored at −80° C. Muscle fiber oxidative capacity was determined in transverse cryosections as a function of succinate dehydrogenase (SDH) enzymatic staining. Briefly, samples were incubated at 37° C. in darkness for 20 min in a medium containing 37 mM sodium phosphate buffer, 74 mM sodium succinate, and 0.4 mM tetranitroblue tetrazolium.

Immunohistochemistry.

Serial transverse cryosections (9 μm thick at intervals of 90 μm) were obtained from the midbelly of the TA and gastrocnemius isolated from hind limbs of C57B1/6J, mdx, and mdx-tg mice. Frozen muscle sections were processed for isolectin immunohistochemical staining using biotinylated isolectin B4 (Vector Laboratories, Burlingame, Calif., USA). Fiber typing was performed by immunohistochemical staining of myosin heavy chain (MHC) type I, IIA, IIX, and IIB using the mouse monoclonal antibodies A4.840, A4.74, 6.H1, and BF-F3, respectively (Developmental Studies Hybridoma Bank, Iowa City, Iowa, USA). The following antibodies were used for detecting laminin and DAG components: laminin (L9393; Sigma, St. Louis, Mo., USA), dystrophin (D8168; Sigma), utrophin (NCL-DRP2; Novocastra, Wetzlar, Germany), and α/β sarcoglycans (Novocastra). All primary antibodies were visualized using suitable Alexa Fluor secondary antibodies from Molecular Probes (Eugene, Oreg., USA). Isolectin was visualized by a DyLight 488 streptavidin conjugate (Vector Laboratories) according to the supplier instructions. Negative control staining by omitting either the primary or the secondary antibody was included in all studies.

Digital Image Analysis and Morphometrics.

Sections were examined using a Zeiss Axioimager fluorescence microscope, and images were captured using an Axiocam digital camera (Carl Zeiss, Oberkochen, Germany). Digital image evaluation of muscle transverse sections was performed with the public domain ImageJ program (NIH, Bethesda, Md., USA).

Protein Expression by Western Blotting and ELISA.

Frozen gastrocnemius muscles from C57B1/6J, mdx, and mdx-tg mice were pulverized by pestle and mortar, lysed, and immunoblotted. The following antibodies were used: myoglobin (Mb; sc-25607; Santa Cruz Biotechnology, Santa Cruz, Calif., USA), cytochrome c (CytC; 556433; BD Pharmingen, San Diego, Calif., USA), and α-tubulin (ab6046; Abcam, Cambridge, Mass., USA). Secreted Vegfa concentration in muscle lysates was measured using ELISA (R&D Systems, Minneapolis, Minn., USA).

Mitochondrial DNA Isolation and Quantification.

Total DNA was prepared from gastrocnemius and quadriceps from C57B1/6J, mdx, and mdx-tg mice and digested with 100 μg/ml RNase A for 30 min at 37° C. The relative copy numbers of mitochondrial and nuclear genomes from 1 ng total DNA were determined by quantitative PCR (qPCR) with primers specific to mitochondrial Cytb and nuclear Rn18s genes. Serial dilutions of pooled DNA were analyzed in parallel to establish a standard curve.

Gene Expression.

Quadriceps, TA and soleus from both hind limbs of C57B1/6J or C57B1/10J, mdx, and mdx-tg mice were used for studying gene expression. Total RNA was prepared from skeletal muscles using the RNeasy Mini Kit (Qiagen, Valencia, Calif., USA). Total RNA (5 μg) was reverse-transcribed to cDNA with SuperScript II Reverse Transcriptase (Invitrogen, Carlsbad, Calif., USA) and analyzed by qPCR on an AB17900 cycler (Applied Biosystems, Foster City, Calif., USA), using the Applied Biosystems SYBR Green PCR Master Mix. Primers were designed using the software Primer Express 3.0 (Applied Biosystems). All data were normalized to cyclophilin and hprt.

PCR-Based Gene Array.

PCR arrays were carried out using the Skeletal Muscle: Myogenesis and Myopathy PCR Array (cat no. PAMM-0993; SA Biosciences, Frederick, Md., USA). The array profiles the expression of 84 genes involved in skeletal muscle function and disease. Briefly, total RNA was extracted from the TA isolated from the C57B1/6J, mdx, and mdx-tg mice using Trizol reagent (Life Technologies Gaithersburg, Md., USA). RNA from n=3 mice/group was pooled such that there were 3 biological replicates/group and was further processed to obtain cDNA. The cDNA was subjected to the PCR array, as per manufacturer's instructions (SA Biosciences) for the ABI Prism 7900 HT sequence detection system. PCR-array data were analyzed using the online data analysis software from the manufacturer's website to identify gene expression changes (see worldwide web address sabiosciences.com/perarraydataanalysis.php). Four housekeeping genes, i.e., HSP90ab1, ACTB, β-2 microglobulin, and β-glucouronidase, were used to normalize the data. Fold up- or down-regulation of individual genes was calculated for mdx and mdx-tg groups with reference to C57B1/6J control group.

Spontaneous Ambulatory Activity.

Basal spontaneous ambulatory activity was measured by indirect calorimetry. Mice were individually housed in chambers of a comprehensive laboratory animal monitoring system (CLAMS; Columbus Instruments, Columbus, Ohio, USA). Food and water were provided ad libitum. Mice were acclimated in the chambers for 48 h before data collection. The ambulation results were quantified in the number of times different infrared beams were broken during an interval.

Postexercise Fatigue Recovery.

Sensitive transmitters (PDT-4000 G2 E-Mitter sensors; Respironics, Inc. Murrysville, Pa., USA) were attached at the animals' tail for monitoring postexercise activity. Signals emitted by the E-Mitter transponders were sensed by a receiver positioned underneath the animal's housing cage and analyzed using VitalView software (Respironics). Locomotor activity counts were recorded in 1-min intervals. Data were reported for 1 h postexercise as cage movements, defined as the frequency of cage movements over time.

Statistical Analysis.

Data are presented as means±sd. Significant differences between groups (C57B1/6J or C57B1/10J, mdx, and mdx-tg) for dependent variables were determined by using 1-way ANOVA followed by Bonferroni's multiple comparison tests. Blood CK levels for mdx and mdx-tg pre- and postexercise were analyzed by a 2-way (genotype exercise) ANOVA. Wherever applicable, comparison between 2 groups was performed by Student's t test for independent variables. Differences were considered statistically significant at P<0.05.

### Example 2

### Metabolic and Angiogenic Gene Deficit in Dystrophic Mice

This example demonstrates metabolic and angiogenic gene deficit in dystrophic mice.

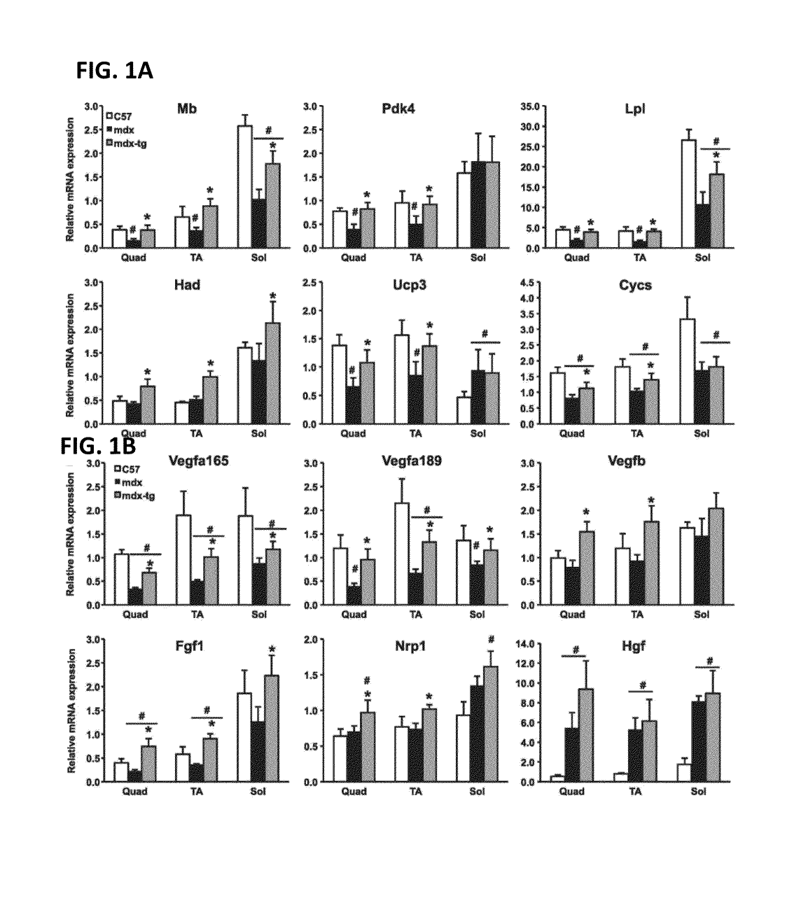

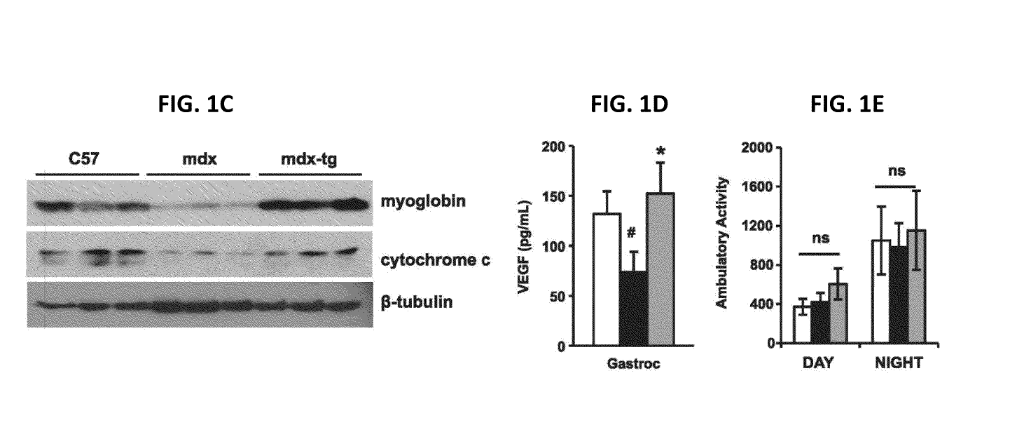

The potential loss of oxidative and vascular capacity, and especially the expression of genes encoding the metabolic and angiogenic features, is not well characterized in DMD. It was asked whether the expression of key genes in this category is affected in the skeletal muscle beds of mdx mice, a preclinical model of DMD. The inventors found that the expression of candidate oxidative metabolic genes [such as Mb, pyruvate dehydrogenase kinase 4 (Pdk4), lipoprotein lipase (Lpl), uncoupling protein 3 (Ucp3), and CytC (Cycs)] as well as angiogenic genes [VEGF-A (Vegfa165, Vegfa189) and FGF1 (Fgf1)] were significantly down-regulated in mdx compared with age-matched C57B1/6J muscles (FIGS. 1A, 1B). The repression of the gene expression program is also evident at the protein level, as the expression of biomarkers such as myoglobin, cytochrome c, and VEGF is reduced in the mdx skeletal muscle (FIGS. 1C, 1D). Nuclear receptor ERRγ is a powerful regulator of oxidative metabolism and vasculature in the skeletal muscle. Therefore, it was determined whether the expression of ERRγ (Esrrg) was reduced in the dystrophic compared with the C57B1/6J skeletal muscle. Indeed, the endogenous ERRγ gene expression was 60-85% lower in the muscle beds of the mdx mouse compared with C57B1/6J mice. Interestingly, gene expression of the functionally related receptor ERRα (Esrra) as well as ERRβ (Esrrb) was also significantly reduced in dystrophic muscles compared with C57B1/6J muscles, demonstrating that endogenous ERR activity was repressed in dystrophin deficient mice. In examining factors related to ERRs, PGC1α (Ppargc1α), a known coactivator of ERR and an antidystrophic target, was repressed in mdx compared with C57B1/6J muscles. This was not the case with GA-binding protein transcription factor-α (Gabpa), which was activated by PGC1α in mitigating dystrophy. Note that the above changes in gene/protein expression between mdx and C57B1/6J muscle were not caused by differences in ambulation between the two groups of mice (FIG. 1E). To further confirm these findings, the expression of selective biomarker genes in TA isolated from mdx mice were compared to those of an alternative wild-type control, the C57B1/10J mice. Similar to the above results, the expression of both the metabolic (Mb, Pdk4, Lpl) and angiogenic (Vegfa165 and Vegfa189) genes was decreased in mdx compared with the C57B1/10J muscles. In addition, the expression of metabolic and angiogenic regulators, such as Esrra, Esrrg, and Ppargc1a, were also repressed in mdx compared with C57B1/10J muscles. Since the gene expression changes in mdx vs. C57B1/6J mice and mdx vs. C57B1/10J mice were similar, age-matched C57B1/6J mice were used as controls for all subsequent studies. The controls are referred to as wild-type mice henceforth. Because loss of ERRγ might potentially contribute to the repression of metabolic and angiogenic genes, it was determined whether selective induction of ERRγ in the skeletal muscles of mdx mice, via transgenesis, restored the repressed gene expression. To do so, the previously described muscle-specific ERRγ transgenic mice were bred with the mdx mice to obtain the mdx (control) and mdx-tg (muscle-specific ERR_-overexpressing) mice. Transgenic induction of ERRγ in the skeletal muscles of mdx mice resulted in the restoration of both oxidative and angiogenic genes that were repressed in muscular dystrophy (FIGS. 1A, 1B). Similarly, the expression of biomarker proteins such as Mb, CytC, and VEGF was also restored by ERRγ in the mdx skeletal muscle (FIGS. 1C, D). ERRγ also induced other muscle genes [e.g., 3-hydroxy acyl-CoA dehydrogenase (Had), VEGF-B (Vegfb), neuropilin 1 (Nrpl), and hepatocyte growth factor (Hgf)] that were not necessarily repressed in the dystrophic mice, but might contribute to metabolic and angiogenic remodeling. The expression of Esrrb (but not Esrra and Ppargc1a) was restored by ERRγ transgenesis. The gene expression changes brought about by the ERRγ were not linked to any difference in ambulatory activity between mdx-tg and mdx mice (FIG. 1E).

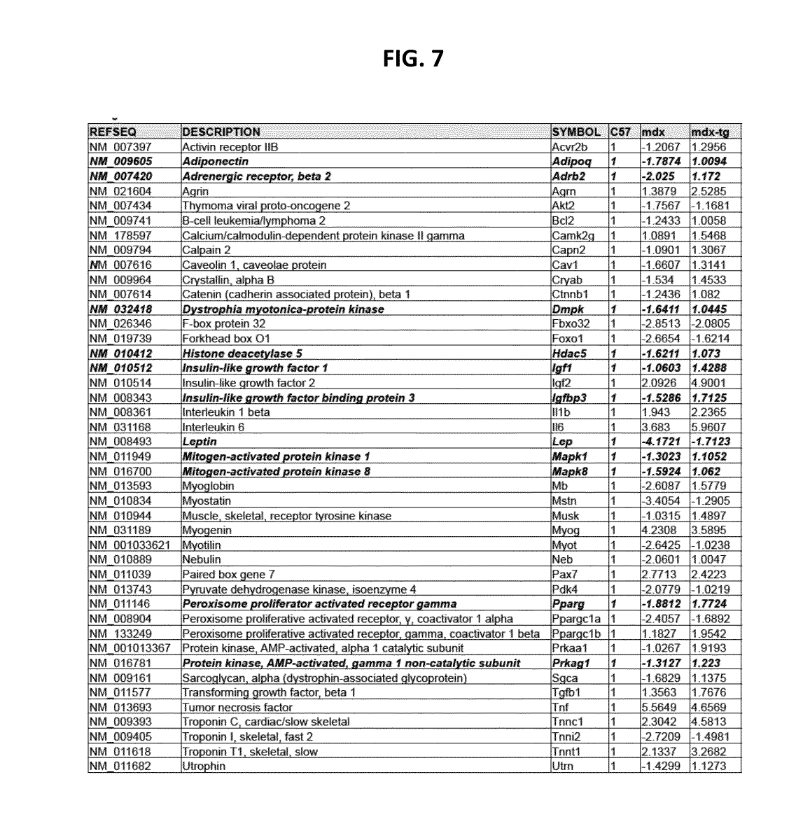

A PCR-based gene array specifically designed to profile the expression of genes linked to myogenesis and myopathy was employed. It was found that the expression of various genes encoding transcription factors (e.g., Pparg and Hdac5), growth factors (e.g., Igfl), cell signaling factors (e.g., Adrb2, Dmpk, Igfbp3, Mapk1, Mapk8, and Prkagl), and other secretory factors (e.g., Adipog and Lep) that might be involved in muscle regeneration were downregulated in mdx vs. wild-type TA muscle. Several of these genes (but not all) were restored by ERRγ overexpression in the dystrophic muscle. The graphical and tabular presentation as well as the details of the target genes is provided in FIG. 7. These data indicate that in addition to metabolic and angiogenic genes, ERRγ may also target and restore other pathways affected in DMD.

### Example 3

### Reprogramming Oxidative Capacity in Dystrophic Muscle

This example demonstrates reprogramming oxidative capacity in dystrophic muscle.

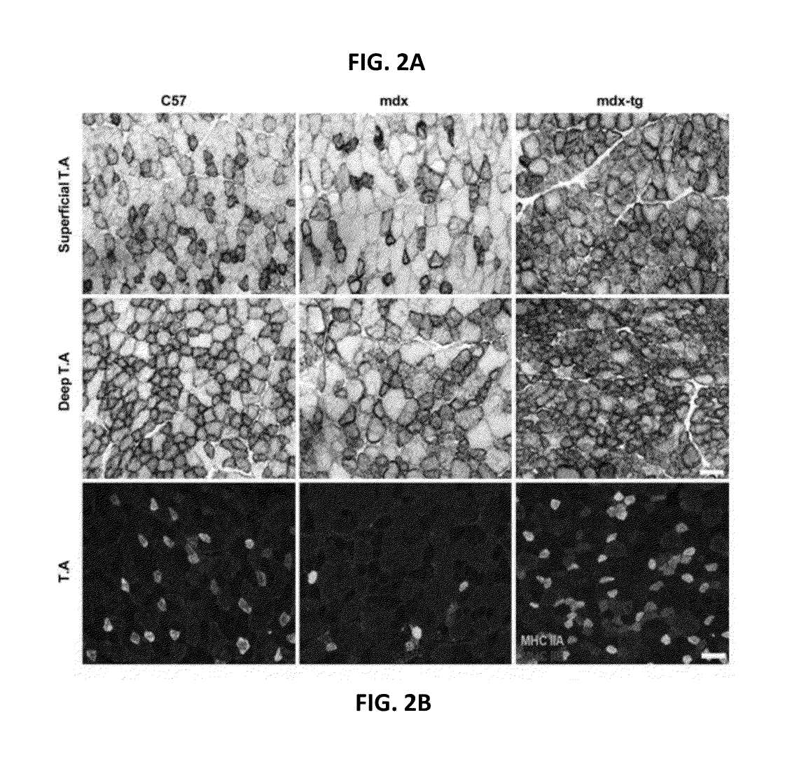

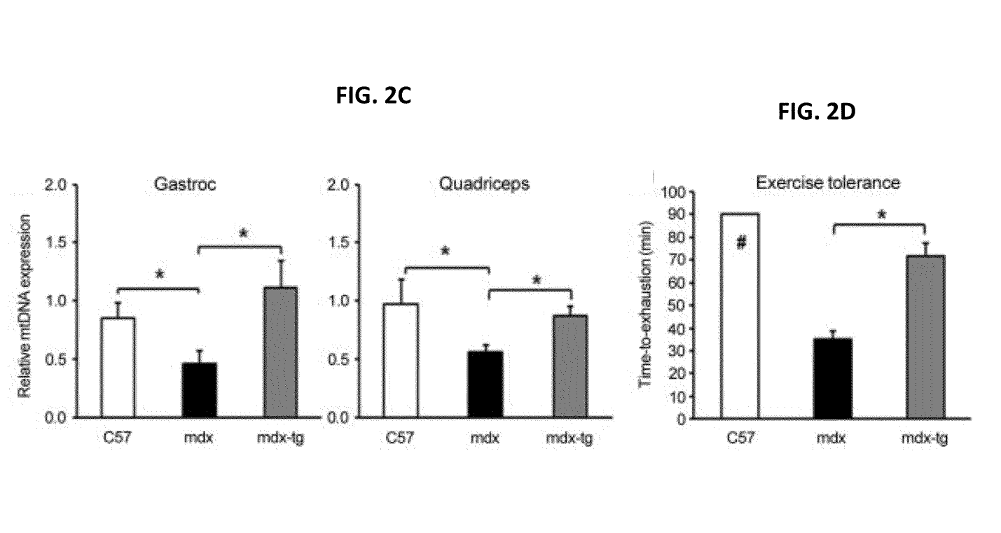

The metabolic and angiogenic gene deficit points toward a compromised oxidative myofiber capacity and/or vascular supply in the dystrophic skeletal muscle. Therefore, aerobic hallmarks such as oxidative capacity (determined by SDH), fiber type composition (MHC staining), and mitochondrial biogenesis (qPCR), as well as exercise tolerance, were measured in the wild-type, mdx, and mdx-tg mice. Enzymatic SDH staining revealed that oxidative capacity was reduced in both the superficial and deep regions of dystrophic compared with the wild-type TA (FIG. 2A). This loss was striking in the deep TA, which typically expresses more oxidative myofibers. Transgenic overexpression of ERRγ restored muscle SDH activity in the mdx-tg compared with mdx mice (FIG. 2A).

Muscle oxidative capacity is in part determined by the proportions of mitochondria-rich myofibers, such as type I, IIA, and IIX. In examining the TA, which is composed of type IIA, IIB, and IIX myofibers, we found that muscular dystrophy resulted in the loss of type IIA and IIX myofibers, which are oxidative in nature. This is shown in FIG. 2B (depicting central TA). Similar to SDH staining, ERRγ overexpression resulted in an increase in type IIA and IIX myofibers in the dystrophic muscle (FIG. 2B). There was no change in type IIB myofibers in mdx compared with wild-type muscles. Moreover, type IIB myofibers were replaced in mdx-tg muscles by oxidative myofibers. The lack of change in type IIB myofibers in the mdx muscles, despite loss of type IIA and IIX myofiber, was not linked to the induction of type I myofibers, but might be due to increase in developmental myofibers seen in dystrophic muscles due to constant degeneration and regeneration. In concert with the above immunohistological changes, mitochondrial biogenesis (determined by the quantification of mtDNA) was found to be reduced in the mdx compared with wild-type muscles (FIG. 2C). ERRγ overexpression increased muscle mitochondrial biogenesis in the mdx-tg mice compared with mdx mice. To determine whether the observed metabolic changes in the dystrophic muscle affect exercise tolerance, the aforementioned mice were subjected to forced exercise via a maximum swimming test. The swimming capacity was significantly reduced in the mdx mice compared with the wild-type mice. Muscle-specific ERRγ overexpression increased the tolerance to endure swimming exercise in the mdx-tg compared with mdx mice (FIG. 2D).

### Example 4

### Reprogramming Vascular Supply in Dystrophic Muscle

This example demonstrates reprogramming vascular supply in dystrophic muscle.

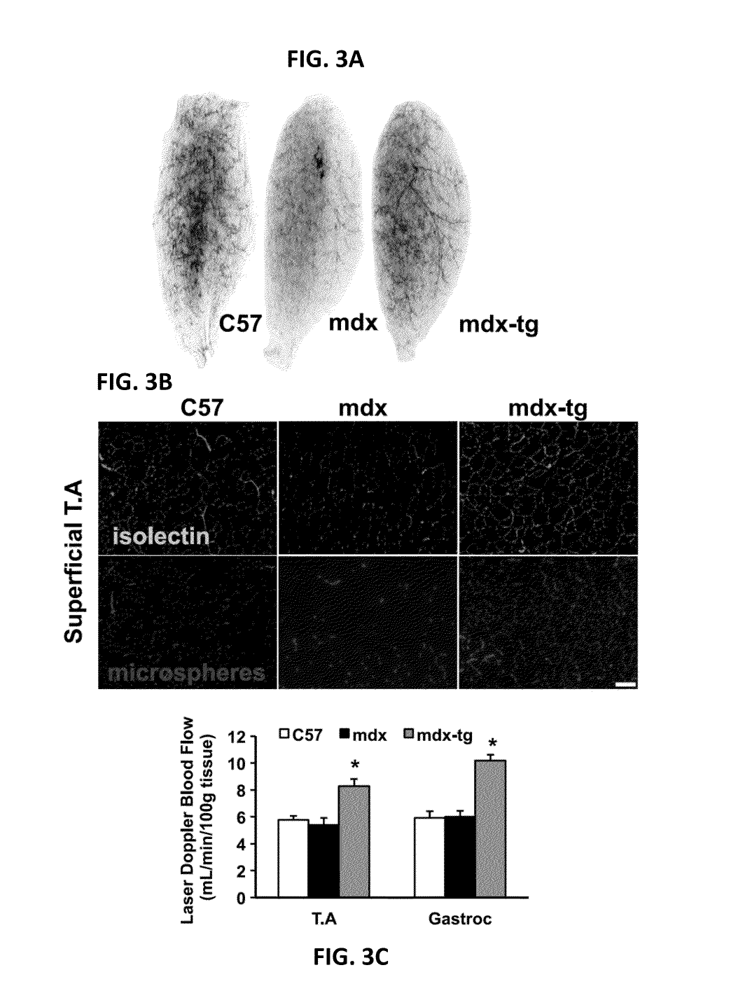

The vascular status in the dystrophic muscle was determined by microfil and microsphere-based muscle vascular mapping, isolectin-assisted staining of vascular structures, and laser Doppler blood flowmetry. Whole mount imaging of mircofil-perfused wild-type and mdx TA revealed that the vasculature was reduced in the dystrophic skeletal muscle (FIG. 3A). Transgenic overexpression of ERRγ reversed the vascular insufficiency in the skeletal muscles of mdx-tg compared with mdx mice. Immunohistology of TA to mark capillary structures using isolectin staining showed a deficit in muscle angiogenesis in mdx vs. wild-type muscle, which was corrected by ERRγ overexpression in mdx-tg muscles (FIG. 3B, top panels). In further support, examination of fluorescent microsphere-perfused muscle cryosections yielded similar results (FIG. 3B, bottom panels). Surprisingly, and despite the loss of vasculature, laser Doppler flowmetry could not detect a decrease in blood flow to dystrophic TA or gastrocnemius muscles. This might be linked to the sensitivity of laser Doppler flowmeter in detecting potentially localized changes in blood flow in mdx compared with wild-type muscles. Note that microsphere angiography, which marks functional blood vessels, indeed demonstrates decrease in vascular supply in mdx vs. wild-type muscles (FIG. 3B, bottom panels). Nevertheless, laser Doppler flowmetry was able to detect an improved blood flow in muscles of mdx-tg compared with mdx mice (FIG. 3C).

### Example 5

### ERRγ Mitigates Dystrophic Pathology in mdx Mice

This example demonstrates ERRγ mitigates dystrophic pathology in mdx mice.

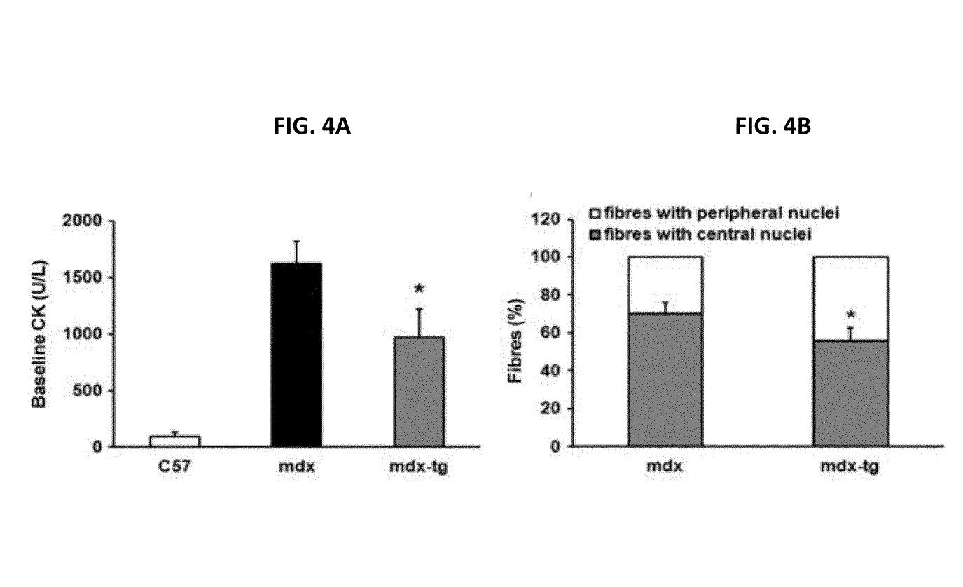

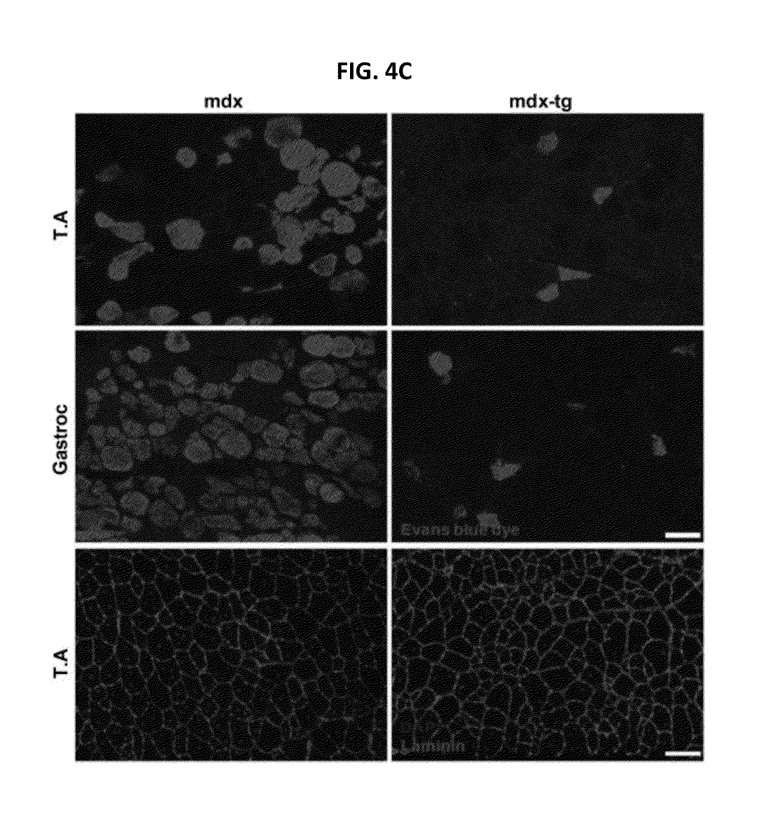

Increase in metabolic, angiogenic, and exercise potential by muscle ERRγ overexpression raised the possibility that the receptor might have mitigated development of dystrophic pathology in the mdx mice. Therefore, the effects of ERRγ overexpression on dystrophic pathology in the mdx mice were determined by using serum CK measurement as well as EBD infiltration in the muscle as diagnostic markers for muscular damage. Although the serum CK levels were higher in mdx mice compared with the wild-type mice, transgenic overexpression of ERRγ specifically in the dystrophic skeletal muscle significantly lowered serum CK in the mdx-tg compared with mdx mice (FIG. 4A). Note that the CK levels in the mdx-tg were not reduced to levels similar to the wild-type mice, which might be due to the process of continuous degeneration despite enhanced regeneration in dystrophic muscle. Nevertheless, EBD infiltration test to mark damaged myofibers showed a dramatic decrease in damaged myofibers in TA and gastrocnemius of mdx-tg compared with mdx mice (FIG. 4B). Similar results were also obtained in other muscles, such as plantaris and diaphragm. In addition, there was a decrease in the number of fibers with centrally located nuclei and a concomitant increase in fibers with peripheral nuclei in the TA of mdx-tg compared with the mdx mice (FIG. 4C, 4D). These findings collectively demonstrate a rescue of dystrophic pathology in mdx mice by muscle-specific ERRγ overexpression.

### Example 6

### ERRγ Prevents Postexercise Damage in Dystrophic Skeletal Muscle

This example demonstrates ERRγ prevents postexercise damage in dystrophic skeletal muscle.

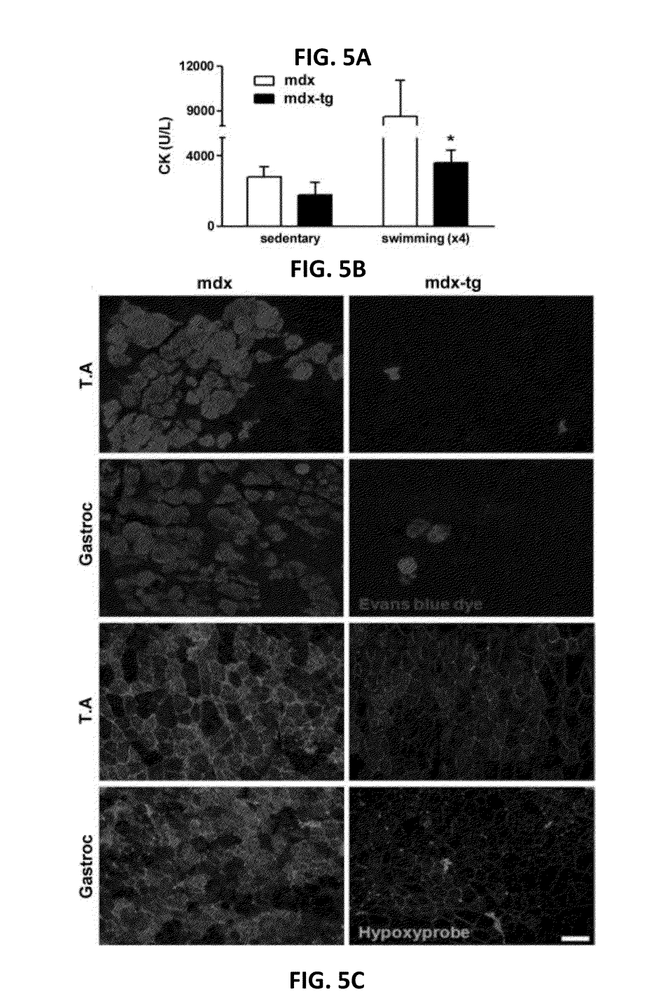

DMD is characterized by susceptibility to exercise-induced muscle damage, as well as post-exercise fatigue, especially due to exaggerated vascular insufficiency and hypoxia. Because ERRγ overexpression improved metabolic and angiogenic features, as well as ameliorated basal dystrophic pathology, it was determined whether the receptor might similarly dampen exercise induced muscle damage in mdx mice. The mdx and mdx tg mice were subjected to swimming exercise bouts of 30-min to determine the extent to which ERRγ protects against post-exercise damage in dystrophic muscle. Serum CK levels were measured pre-exercise and 1 h post-exercise in mdx and mdx-tg mice after 30-min bouts of swimming per day conducted on 4 consecutive days. This exercise protocol induced muscle damage, as demonstrated by a dramatic increase in serum CK in mdx/exercise compared with mdx/sedentary mice (FIG. 5A). However, the exercise-induced increase in serum CK was suppressed in mdx-tg mice, suggesting that ERRγ might protect against post-exercise damage. This was evaluated by using the EBD infiltration test. Significantly fewer EBD-positive myofibers were found in the muscles of mdx-tg compared with mdx mice in response to exercise, confirming that ERRγ protects against post-exercise muscle damage (FIG. 5B). As mentioned above, post-exercise muscle damage in mdx mice is partly linked to vascular insufficiency and resulting hypoxia/ischemia in the skeletal muscle. Data described above show that ERRγ triggers a robust angiogenic reprogramming in the dystrophic skeletal muscle. Therefore, whether the exercise-induced hypoxia in the dystrophic muscle was mitigated by ERRγ was investigated. Hypoxia was found (detected using hypoxyprobe) in dystrophic TA and gastrocnemius muscles of the post-exercised mdx mice. Indeed, this post-exercise muscle hypoxia was prevented by ERRγ in the mdx-tg mice (FIG. 5C). Similar findings were obtained in other muscles groups that are active during exercise (e.g., triceps brachii and EDL). Note that the levels of hypoxia in nonmuscle control tissues (e.g., liver) were comparable between mdx and mdx-tg mice. A combined effect of post-exercise muscle damage and vascular insufficiency is severe fatigue with pro-longed recovery period, which can be tracked by the latency/lethargy of the mice. In accordance with the ERRγ-dependent aerobic remodeling of the skeletal muscle, 100% of mdx-tg mice recovered from post-exercise fatigue within 60 min after a single 30-min swimming bout. On the contrary, at this time point the mdx mice still exhibited severe fatigue and inability to ambulate. To quantify this phenomenon, the mdx and mdx-tg mice were subjected to activity monitoring cages. Indeed, the rodent activity within 1 h post-exercise was significantly lower in the mdx group compared with mdx-tg mice (mdx vs. mdx-tg: 516±271 vs. 1900±500 cage movements; P<0.001, unpaired Student's t test; n=6).

### Example 7

### ERRγ Fails to Increase Utrophin or Restore DAG Complex

This example demonstrates ERRγ fails to increase utrophin or restore DAG complex.

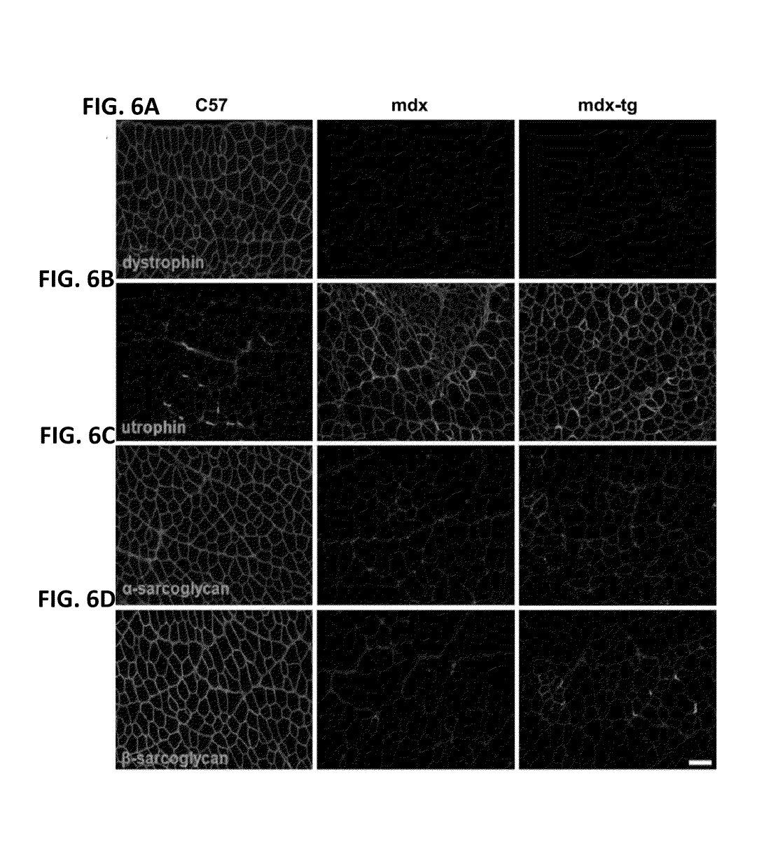

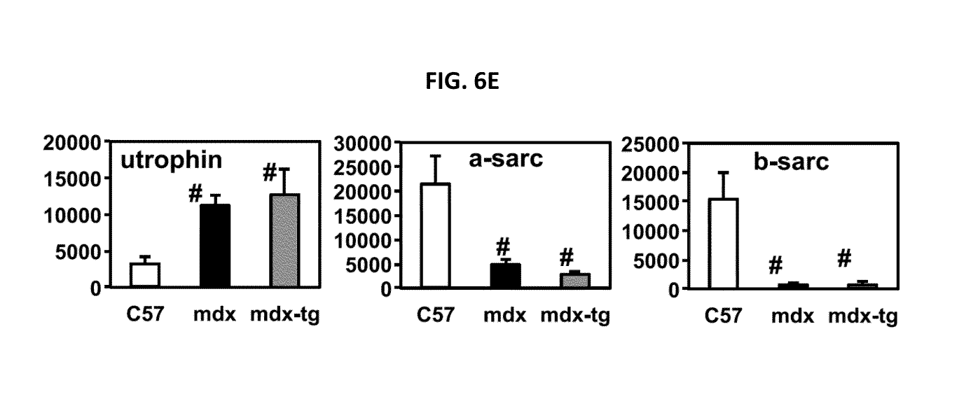

The rescue of basal as well as post-exercise damage in the dystrophic skeletal muscle is akin to one achieved by minidystrophin or utrophin gene replacement. Indeed, previous reports have suggested that oxidative myofibers are enriched in utrophin. Consequently, oxidative transformation in the dystrophic muscle may increase utrophin, which in turn might act as a structural surrogate of dystrophin. Since ERRγ reprogrammed the muscle to an oxidative phenotype in mdx mice, it was determined whether the antidystrophic effects of the receptor might be linked to sacrolemmal enhancement of utrophin and DAG complex. It was shown by immunofluorescence that sarcolemmal utrophin expression was upregulated in the TA of the mdx compared with wildtype mice. To the inventors' surprise and unlike other oxidative myofiber regulators, ERRγ did not further induce sarcolemmal utrophin expression (FIG. 6B, 6E). Additional examination of even the deeper TA, where the metabolic and angiogenic deficit was found to be greater, did not show any induction of sarcolemmal utrophin by ERRγ. The lack of a major effect of ERRγ on utrophin was further confirmed by demonstrating that the utrophin gene expression was only modestly induced by the receptor. Consequently, ERRγ did not result in sarcolemmal restoration of the components of the DAG complex (as exemplified by membrane α/β-sarcoglycan expression), typically lost from the dystrophic membrane (FIGS. 6C-6E). Sarcolemmal dystrophin staining is shown as control (FIG. 6A).

In summary, it was shown that ERRγ, its metabolic and angiogenic gene targets, oxidative capacity, and vasculature are repressed in muscular dystrophy. Transgenic induction of ERRγ restores the aforementioned features, as well as mitigates both the basal and exercise induced dystrophic pathology. Notably, and distinct from other metabolic regulators, ERRγ ameliorates dystrophy without sarcolemmal enhancement of utrophin or restoration of the components of DAG complex.

DMD is a fatal disorder with poor treatment options. Here, it is shown that oxidative capacity, mitochondrial biogenesis, and vascularity, as well as exercise tolerance, are abated in the skeletal muscles of the mdx mice. This metabolic and angiogenic muscle pathology is associated with repressed expression of nuclear receptor ERRγ, related receptors ERRα and ERRβ, and ERR-regulated metabolic and angiogenic genes. Transgenic induction of ERRγ selectively in the muscles of mdx mice restored the metabolic and angiogenic deficit and mitigated basal and exercise-induced muscle damage. Interestingly, ERRγ retarded dystrophy without inducing utrophin or restoring sarcolemmal DAG complex. Therefore, ERRγ is a signaling node affected by dystrophin deficiency, targeting of which can mitigate DMD in absence of utrophin induction or membrane DAG restoration. While dystrophin deficiency in DMD causes structural instability by increasing susceptibility to sarcolemmal damage and myofiber rupture, its loss is also linked to deregulation of multiple cellular signaling pathways. For example, leaky calcium channels, protease activation, oxidative stress, microtubule dysfunction, impaired ATP production, and defective nitric oxide signaling contribute to the pathology of DMD. The present results show that DMD was also associated with a deficit in oxidative metabolic capacity, mitochondrial biogenesis, oxidative myofibers (type IIA and IIX), and angiogenesis in the skeletal muscle; overall, resulting in aerobic deficiency as indicated by exercise intolerance in the mdx mice. While impaired mitochondrial respiration and oxidative phosphorylation were recently reported in the muscles isolated from the mdx mice, widespread fiber type deregulation and vascular regression in muscular dystrophy were previously less appreciated. The aerobic deficiency in the dystrophic muscles was linked to a coordinated down-regulation of both oxidative metabolic and angiogenic genes. It is suggested herein that the dysfunctional metabolic and angiogenic gene program is partly linked to decrease in the expression of regulators such as ERRα, ERRβ, and ERRγ.

ERRβ was also found to be down-regulated in dystrophic muscles. The role of this receptor in the skeletal muscle function has not previously been investigated. Interestingly, other metabolic regulators, such as PGC1α, PPARγ, PPARγ, and AMPK (γδ-regulatory subunit), were also found to be downregulated in dystrophic muscle. The repression of these factors may also be responsible for the metabolic and angiogenic deficit in the mdx muscle. Therefore, ERRγ and related nuclear receptors as well as its coactivators may be components of a metabolic and angiogenic transcriptional circuitry in muscle that is repressed in DMD, restoration of which mitigates dystrophic pathology. Overexpression of the dystrophin homologue utrophin in the skeletal muscle improves sarcolemmal integrity and stability by substituting for dystrophin and restoring DAG complex in the dystrophic mice. Interestingly, utrophin is highly expressed in the oxidative slow-twitch muscles and is induced by the molecular regulators of oxidative slow myogenic program. Surprisingly, a glycolytic-to-oxidative fiber type shift by ERRγ in the dystrophic muscle did not result in increased sarcolemmal localization of utrophin in the mdx mice. Note that a modest increment in utrophin gene expression was observed by ERRγ in dystrophic muscle, which did not culminate in utrophin localization in the sarcolemma. In support of this, typical components of the sarcolemmal DAG complex were not restored by ERRγ in the mdx mice, as would be by utrophin induction. In the context of this finding, how might ERRγ mitigate DMD without inducing utrophin or restoring DAG complex? One mechanism could be via restoration of neuronal nitric oxide synthase (nNOS) and nitric oxide signaling in the dystrophic skeletal muscle. Alternatively, muscle ERRγ could boost muscle regeneration in mdx mice resulting in muscle repair. The present finding that ERRγ decreases the number of myofibers with centrally located nuclei, while concomitantly increasing myofibers with peripheral nuclei in mdx mice is indicative of advanced regeneration. Quite remarkably, ERRγ prevented post-exercise damage and fatigue in the mdx mice. The post-exercise pathology in DMD is associated with mitochondrial dysfunction and unattenuated vasoconstriction in the muscle beds. Histological examination of mdx muscles post-exercise has revealed extended areas with vascular narrowing, which results from the loss of nNOS. nNOS has been shown to increase blood flow in post-exercise muscles by blunting sympathetic vasoconstriction in contracting muscles. However, the vasodilatory mechanism is absent in dystrophy due to loss of muscle nNOS resulting in ischemic damage, which contributes to the pathogenesis of DMD. Because ERRγ neither increased utrophin nor restored the DAG components, it is believed that the resilience of transgenic mdx muscle to post-exercise damage and fatigue is imparted by improved oxidative capacity but more importantly by enhanced muscle angiogenesis. It was found that exercise-induced hypoxia in mdx muscle is eliminated by ERRγ, most likely by inducing angiokine expression, capillary density, and muscle oxygenation.

Since the cloning of dystrophin and identification of inactivating mutation in DMD, various therapeutic approaches such as cell-based therapy, gene replacement therapy, and synthetic drugs have aimed toward replacing dystrophin or inducing utrophin. Although promising, cell-based therapy and gene replacement with viral delivery vectors are challenged by immunological rejections and issues related to widespread muscle delivery, whereas current synthetic methods such as exon skipping by antisense oligonucleotides so far have poor bioavailability. In contrast, pharmacological drugs that could induce utrophin or independently combat DMD pathology have the benefit of easy systemic delivery and lack potential immunological reaction. Nuclear receptors are one of the most successful pharmacologically targeted classes of regulators in various diseases. Here, it is shown that ERRγ elicits metabolic and angiogenic remodeling in the dystrophin-deficient skeletal muscle leading to an increase in oxidative capacity, mitochondrial biogenesis, fiber type conversion, as well as augmented capillary density and improved blood flow. It is also shown that ERRγ-driven remodeling rescues dystrophic pathology without requiring sarcolemmal enhancement of utrophin or DAG components.

The present examples demonstrate ERR as a therapeutic target for treating DMD and simultaneously points to the possibility that ERR-metabolic-angiogenic deficiency as a common feature of various type of muscular dystrophies.

### Example 8

### Method of Treating Muscular Dystrophy

This example illustrates the methods of treating DMD via administering an agent including ERRγ agonist, GW4064, or ERRγ-specific agonist/antagonists derived from the GW4064 scaffold.

Based upon the teachings disclosed herein, one or more signs or symptoms of DMD can be reduced or inhibited by administering GW4064. In one specific, non-limiting example, a unit dosage for intravenous or intramuscular administration of an ERRγ agonist (GW4064) includes at least 0.5 μg agonist per dose, such as at least 5 μg agonist per dose, at least 50 μg agonist per dose, or at least 500 μg agonist per dose. In some examples, doses are administered three-times in one week.

In one specific, non-limiting example, an ERRγ agonist (GW4064) daily dosage is from about 0.01 milligram to about 500 milligram per kilogram of animal body weight, for example given as a single daily dose or in divided doses two to four times a day, or in sustained release form. For most large mammals, the total daily dosage is from about 0.01 milligrams to about 100 milligrams per kilogram of body weight, such as from about 0.5 milligram to about 100 milligrams per kilogram of body weight, which can be administered in divided doses 2 to 4 times a day in unit dosage form containing for example from about 10 to about 100 mg of the compound in sustained release form. In one example, the daily oral dosage in humans is between 1 mg and 1 g, such as between 10 mg and 500 mg, 10 mg and 200 mg, such as 10 mg. The dosage regimen may be adjusted within this range or even outside of this range to provide the optimal therapeutic response. Oral administration of an ERRγ agonist can be carried out using tablets or capsules, such as about 10 mg to about 500 mg of the ERRγ agonist. Exemplary doses in tablets include 0.1 mg, 0.2 mg, 0.25 mg, 0.5 mg, 1 mg, 2 mg, 5 mg, 10 mg, 25 mg, 50 mg, 100 mg, 250 mg, and 500 mg of the ERRγ agonist. Other oral forms can also have the same dosages (e.g., capsules). In one example, a dose of an ERRγ agonist administered parenterally is at least 10 mg, such as 10 to 500 mg or 10 to 200 mg of the ERRγ agonist.

In one specific, non-limiting example, a unit dosage for oral administration such as a table or capsule), or for oral intravenous or intramuscular administration, of an ERRγ protein includes about 1 μg to 1000 mg of ERRγ protein per dose, such as 1 μg to 100 μg ERRγ protein per dose, 1 μg to 500 μg ERRγ protein per dose, 1 μg to 1 mg ERRγ protein per dose, 1 mg to 1000 mg ERRγ protein per dose, or 10 mg to 100 mg ERRγ protein per dose. In some examples, doses are administered at least three-times in one week.

In one specific, non-limiting example, a unit dosage for administration of an ERRγ nucleic acid (such as injection, gene gun, pneumatic injection, or topical) includes at least 10 ng, at least 100 ng, at least 1 at least 10 μg, at least 100 μg, or at least 500 μg nucleic acid per dose. Saline injections can use amounts of DNA, such as from 10 μg-1 mg, whereas gene gun deliveries can require 100 to 1000 times less DNA than intramuscular saline injection (such as 0.2 μg-20 μg). These amounts can vary from species to species, with mice, for example, requiring approximately 10 times less DNA than primates. Saline injections may require more DNA because the DNA is delivered to the extracellular spaces of the target tissue (e.g., muscle), where it has to overcome physical barriers before it is taken up by the cells, while gene gun deliveries bombard. DNA directly into the cells.

In one specific, non-limiting example, a unit dosage for intravenous or intramuscular administration of a viral vector that encodes ERRγ includes at least 1×10⁸ viral particles per dose, such as at least 1×10⁹ viral particles per dose, at least 1×10¹⁰ viral particles per dose, or at least 1×10¹¹ viral particles per dose.

In one specific example, the effects of treatment with the ERRγ agonist GW4064 would be determined by improvements in handgrip strength, exercise capacity, 02 consumption/workload, 3 or 6 minute walk test, non-invasive Cardio-pulmonary exercise test (CPET-measuring cardiac output, maximum volume of oxygen consumption (VO₂) and carbon dioxide output (VCO₂), minute ventilation (VE), and maximum voluntary ventilation (MVV)) and invasive CPET (intracardiac hemodynamic and arterial blood gas data generated during exercise from a pulmonary artery and radial catheter). Effective treatment would be indicated by a 20% improvement in any of the above measured parameters.

### Example 9

### Screening of Agents to Modulate ERRγ

This example describes methods that can be used to identify agents to modulate ERRγ, such as to treat muscular dystrophy.

According to the teachings herein, one or more agents for modulating ERRγ can be identified by contacting a cell, such as a cell expressing ERRγ, with one or more test agents under conditions sufficient for the one or more test agents to alter the activity of ERRγ. In one particular example, the method is for identifying an ERRγ agonist. An increase in the binding of the synthetic ligand of ERRγ relative to a control identifies the agent as one that is useful to enhance muscle regeneration and/or treat muscular dystrophy. Increased binding is detected by use of the commercially available LanthaScreen Estrogen Related Receptor gamma TR-FRET co-activator assay (INVITROGEN®). Agents that cause at least a 2-fold increase, such as at least a 3-fold increase, at least a 4-fold increase, or at least a 5-fold increase in the activity are selected for further evaluation.

Potential therapeutic agents identified with these or other approaches, including the specific assays and screening systems described herein, are used as lead compounds to identify other agents having even greater modulatory effects on ERRγ. Candidate agents also can be tested in additional cell lines and animal models of muscular dystrophy to determine their therapeutic value. The agents also can be tested for safety in animals, and then used for clinical trials in animals or humans. In one example, genetically engineered mouse models of muscular dystrophy are employed to determine therapeutic value of test agents.

In view of the many possible embodiments to which the principles of our invention may be applied, it should be recognized that illustrated embodiments are only examples of the invention and should not be considered a limitation on the scope of the invention. Rather, the scope of the invention is defined by the following claims. We therefore claim as our invention all that comes within the scope and spirit of these claims.

We claim:

1. A method of enhancing muscle regeneration, repair, or maintenance in a subject comprising administering an effective amount of an agent that increases expression or activity of estrogen receptor-related gamma (ERRγ), related receptors ERRα or β, or ERR-regulated metabolic and angiogenic genes, thereby enhancing muscle regeneration, maintenance, or repair, thereby enhancing muscle regeneration, repair, or maintenance in a subject.

2. The method of claim 1, wherein the subject has one or more signs or symptoms of a muscular dystrophy.

3. The method of claim 1, wherein the method is used to enhance muscle regeneration, repair, or maintenance in a subject with Duchenne muscular dystrophy.

4. The method of claim 1, further comprising selecting a subject in need of enhancing muscle regeneration, maintenance, or repair.

5. The method of claim 4, wherein selecting a subject in need of enhancing muscle regeneration, maintenance, or repair comprises detecting the expression or activity of ERRγ, related receptors ERRα or β, or ERR-regulated metabolic and angiogenic genes in a biological sample, wherein detecting a decreased expression level of one or more of ERRγ, related receptors ERRα or β, or ERR-regulated metabolic and angiogenic genes indicates the subject is need of enhancing muscle regeneration, maintenance, or repair.

6. The method of claim 5, wherein detecting a decreased expression level of one or more of ERRγ, related receptors ERRα or β, or ERR-regulated metabolic and angiogenic genes indicates the subject is suffering from a muscular dystrophy, such as Duchenne muscular dystrophy (DMD), Becker muscular dystrophy, limb-girdle muscular dystrophy, a congenital muscular dystrophy, facioscapulohumeral muscular dystrophy, myotonic muscular dystrophy, oculopharyngeal muscular dystrophy, distal muscular dystrophy, and/or Emery-Dreifuss muscular dystrophy.

7. The method of claim 1, wherein the agent increases expression or activity of estrogen receptor-related gamma (ERRγ).

8. The method of claim 7, wherein the agent is GW4064, a GW4064 analog, bisphenol A (BPA), GSK 4716, a GSK4716 analog or a combination thereof.

9. The method of claim 7, wherein the agent is GW4064.

10. A method of treating a muscular disease or demyelinating disease, comprising:
administering an effective amount of an agent that increases the expression or activity of estrogen receptor-related gamma (ERRγ), related receptors ERRα or β, or ERR-regulated metabolic and angiogenic genes to a subject with one or more signs or symptoms of a muscular disease, such as muscular dystrophy or a demyelinating disease, thereby treating one or more signs or symptoms associated with the muscular, such as muscular dystrophy, or demyelinating disease in the subject.

11. The method of claim 10, wherein the muscular dystrophy is Duchenne muscular dystrophy (DMD), Becker muscular dystrophy, limb-girdle muscular dystrophy, congenital muscular dystrophy, facioscapulohumeral muscular dystrophy, myotonic muscular dystrophy, oculopharyngeal muscular dystrophy, distal muscular dystrophy, and/or Emery-Dreifuss muscular dystrophy or the demyelinating disease is multiple sclerosis, Charcot-Marie-Tooth disease, Pelizaeus-Merzbacher disease, encephalomyelitis, neuromyelitis optica, adrenoleukodystrophy, and/or Guillian-Barre syndrome.

12. The method of claim 10, further comprising selecting a subject with one or more signs or symptoms of a muscular disease or demyelinating disease.

13. The method of claim 12, wherein selecting a subject with one or more signs or symptoms of a muscular disease or demyelinating disease comprises detecting the expression or activity of ERRγ, related receptors ERRα or β, or ERR-regulated metabolic and angiogenic genes in a biological sample, wherein detecting a decreased expression level of one or more of ERRγ, related receptors ERRα or β, or ERR-regulated metabolic and angiogenic genes indicates the subject is need of enhancing muscle regeneration, maintenance, or repair.

14. The method of claim 10, wherein the agent increases expression or activity of estrogen receptor-related gamma (ERRγ).

15. The method of claim 14, wherein the agent is GW4064, a GW4064 analog, bisphenol A (BPA), GSK 4716, a GSK4716 analog or a combination thereof.

16. A method for treating a muscular dystrophy, comprising:
contacting a cell with an effective amount of an agent comprising an ERRγ agonist, wherein the agent specifically increases the activation or expression of ERRγ,
thereby treating one or more signs or symptoms associated with a muscular dystrophy.

17. The method of claim 16, wherein the cell is in vivo and the method further comprises a subject in need of treating one or more signs or symptoms associated with a muscular dystrophy and administered of the ERRγ-specific chemical ligand to the subject.

18. The method of claim 16, wherein the cell is in vitro.

19. The method of claim 16, wherein the agent is GW4064, a GW4064 analog, bisphenol A (BPA), GSK 4716, a GSK4716 analog or a combination thereof.

20. A method of determining if an agent is useful to enhancing muscle regeneration, repair, or maintenance in a subject, comprising:
contacting at least one cell with an agent, wherein the cell expresses at least estrogen receptor-related gamma (ERRγ); and
detecting an increase in the binding of a ligand of the ERRγ relative to a control,
wherein an increase in the binding of the ligand of ERRγ to ERRγ relative to a control identifies the agent as one that is useful to enhancing muscle regeneration, repair, or maintenance.
